(19) United States
(12) Patent Application Publication
(10) Pub. No.: US 2022/0267331 A1
(43) Pub. Date: Aug. 25, 2022

(54) DOPAMINE-B-HYDROXYLASE INHIBITORS

(71) Applicant: BIAL - PORTELA & CA, S.A., São Mamede Do Coronado (PT)

(72) Inventors: Laszlo Kiss, São Mamede Do Coronado (PT); Alexander Beliaev, São Mamede Do Coronado (PT); Tino Rossi, São Mamede Do Coronado (PT); Nuno Palma, São Mamede Do Coronado (PT); Patrício Soares Da Silva, Coronado (PT); Rui Pinto, São Mamede Do Coronado (PT); Francisco Cardona, São Mamede Do Coronado (PT)

(21) Appl. No.: 17/615,987

(22) PCT Filed: Jun. 3, 2020
(86) PCT No.: PCT/PT2020/050022

§ 371 Date: Dec. 2, 2021

(30) Foreign Application Priority Data
Jun. 5, 2019 (GB) 1908044.9

(51) Int. Cl. C07D 487/04

(52) U.S. Cl. CPC C07D 487/04

(57) ABSTRACT

This invention relates to: (a) compounds of formula I (with R₁ to R₅ and A as defined herein) and pharmaceutically acceptable salts or solvates thereof that are useful as dopamine-β-hydroxylase inhibitors; (b) pharmaceutical compositions comprising such compounds, salts or solvates; (c) the use of such compounds, salts or solvates in therapy; (d) therapeutic methods of treatment using such compounds, salts or solvates; and (e) processes and intermediates useful for the synthesis of such compounds.

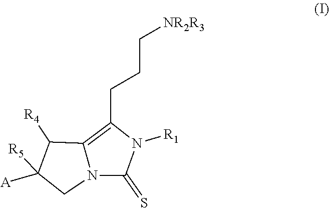

### FIELD OF THE INVENTION

This invention relates to: (a) compounds and pharmaceutically acceptable salts or solvates thereof that are useful as dopamine-p-hydroxylase inhibitors; (b) pharmaceutical compositions comprising such compounds, salts or solvates; (c) the use of such compounds, salts or solvates in therapy; and (d) therapeutic methods of treatment using such compounds, salts or solvates.

### BACKGROUND OF THE INVENTION

The enzyme dopamine-p-hydroxylase (DOH), also known as dopamine (3-monooxygenase, is expressed both in the periphery and the central nervous system (CNS). DβH catalyses the specific hydroxylation of dopamine (DA) to produce norepinephrine, also known as noradrenaline (NA). As such, inhibitors of DβH can inhibit the biosynthesis of NA, limiting its concentration and increasing DA levels.

In recent years, interest in the development of inhibitors of DβH has centred on the hypothesis that inhibition of this enzyme may provide significant clinical improvements in patients suffering from cardiovascular disorders such as hypertension or chronic heart failure. The rationale for the use of DβH inhibitors is based on their capacity to inhibit the biosynthesis of NA, which is achieved via enzymatic hydroxylation of DA. Reduction of the biosynthesis of NA via inhibition of DβH can directly dampen sympathetic nerve function, the activation of which is the principal clinical manifestation of congestive heart failure (Parmley, W. W., Clin. Cardiol., 18: 440-445, 1995). Congestive heart failure patients have elevated concentrations of plasma noradrenaline (Levine, T. B. et al., Am. J. Cardiol., 49:1659-1666, 1982), increased central sympathetic outflow (Leimbach, W. N. et al., Circulation, 73: 913-919, 1986) and augmented cardiorenal noradrenaline spillover (Hasking, G. J. et al., Circulation, 73:615-621, 1966). Prolonged and excessive exposure of the myocardium to noradrenaline may lead to down-regulation of cardiac β₁-adrenoceptors, remodelling of the left ventricle, arrhythmias and necrosis, all of which can diminish the functional integrity of the heart. Congestive heart failure patients who have high plasma concentrations of noradrenaline also have the most unfavourable long-term prognosis (Cohn, J. N. et al., N. Engl. J. Med., 311:819-823, 1984). Of greater significance is the observation that plasma noradrenaline concentrations are already elevated in asymptomatic patients with no overt heart failure and can predict ensuing mortality and morbidity (Benedict, C. R. et al., Circulation, 94:690-697, 1996). An activated sympathetic drive is not therefore merely a clinical marker of congestive heart failure, but may contribute to progressive worsening of the disease.

DβH inhibitors may also display activity the CNS, if they cross the blood-brain barrier (BBB).

Several inhibitors of DβH have been thus far reported in the literature. Early first and second generation examples such as disulfiram (Goldstein, M. et al., Life Sci., 3:763, 1964) and diethyldithiocarbamate (Lippmann, W. et al., Biochem. Pharmacol., 18: 2507, 1969) or fusaric acid (Hidaka, H. Nature, 231, 1971) and aromatic or alkyl thioureas (Johnson, G. A. et al, J. Pharmacol. Exp. Ther., 171: 80, 1970) were found to be of low potency, exhibited poor selectivity for DβH and caused toxic side effects. The third generation of DβH inhibitors, however, were found to have much greater potency, such as, for example, nepicastat (RS-25560-197, IC₅₀ 9 nM) (Stanley, W. C., et al., Br. J. Pharmacol., 121: 1803-1809, 1997), which was developed to early clinical trials. Although it was initially developed for peripheral indications (hypertension and congestive heart failure), an important discovery was that nepicastat was found to cross the BBB, and was thereby able to cause central as well as peripheral effects.

Nepicastat and its analogues are disclosed in WO95/29165. Furthermore, WO 2004/033447 and WO 2008/136695 disclose DβH inhibitors having high potency and significantly reduced brain access, giving rise to potent and peripherally selective DβH inhibitors. However, these compounds are also difficult to synthesise requiring many steps in the synthetic route making them expensive to manufacture. In particular, potent compounds disclosed in WO 2008/136695 are sparingly soluble and display improved levels of exposure when administered with high-fat meals. A review of the mechanism, substrates and inhibitors of DβH, is given by Beliaev, A., et al. in Current Enzyme Inhibition, 5, 27-43, 2009.

WO2018/056854 and WO2018/056855 disclose DβH inhibitors which are useful for the treatment of conditions ameliorated by inhibition of DβH within the CNS. Compared with the compounds of formula I of the present invention, the compounds of WO2018/056854 and WO2018/056855 have different substituents at the 3-position of the fused imidazole ring.

WO2019/112457 (published after the priority date of the present application) discloses DβH inhibitors which are useful for the treatment of conditions ameliorated by inhibition of DβH outside the CNS. Specific compounds disclosed therein include (R)-1-(3-(pyrrolidin-1-yl)propyl)-6-(2,3,5,6-tetrafluorophenyl)-2,5,6,7-tetrahydro-3H-pyrrolo[1,2-c]imidazole-3-thione hydrochloride (Example 219), (R)-1-(3-(pyrrolidin-1-yl)propyl)-6-(2,3,5,6-tetrafluorophenyl)-2,5,6,7-tetrahydro-3H-pyrrolo[1,2-c]imidazole-3-thione (Example 471), and (R)-1-(3-(pyrrolidin-1-yl)propyl)-6-(2,3,6-trifluorophenyl)-2,5,6,7-tetrahydro-3H-pyrrolo[1,2-c]imidazole-3-thione hydrofluoride (Example 478).

Therefore, there remains an unfulfilled clinical requirement for a potent, non-toxic and peripherally selective inhibitor of DβH, which could be used for treatment of certain cardiovascular disorders. A DβH inhibitor with similar or even greater potency than nepicastat, but devoid of CNS effects (i.e. unable to efficiently cross the BBB), yet exhibiting a long residence time in the periphery so as to provide a long duration of DβH inhibition would provide a significant improvement over all DβH inhibitor compounds thus far described in the prior art. Additionally, such compounds would preferably be orally bioavailable, highly soluble and easier and cheaper to synthesise.

### SUMMARY OF THE INVENTION

The present invention provides a compound of formula I, or a pharmaceutically acceptable salt or solvate thereof.

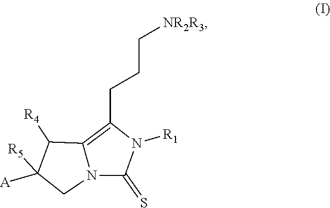

wherein:
R₁ is hydrogen;
R₂ is hydrogen; and
R₃ is hydrogen, methyl, 6-membered heterocyclyl, or CH₂X wherein X is 5- or 6-membered heterocyclyl; or
R₂ is methyl; and
R₃ is methyl, 5- or 6-membered heterocyclyl, or CH₂X wherein X is 5- or 6-membered heterocyclyl; or
R₂ and R₃ combine, together with the N atom to which they are attached, to form a 5- or 6-membered N-heterocyclyl optionally substituted with one fluoro substituent;
R₄ is hydrogen; and
R₅ is hydrogen; or
R₄ and R₅ combine, together with the carbon atoms to which they are attached, to form a cyclopropyl ring; and

### A is

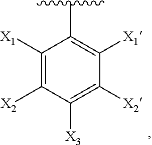 [0012] wherein:
X₁ is hydrogen or halo;
X₁′ is hydrogen or halo;
X₂ is hydrogen or halo;
X₂′ is hydrogen or halo; and
X₃ is hydrogen;
   with the proviso that the compounds
(R)-1-(3-(pyrrolidin-1-yl)propyl)-6-(2,3,5,6-tetrafluorophenyl)-2,5,6,7-tetrahydro-3H-pyrrolo[1,2-c]imidazole-3-thione hydrochloride,
(R)-1-(3-(pyrrolidin-1-yl)propyl)-6-(2,3,5,6-tetrafluorophenyl)-2,5,6,7-tetrahydro-3H-pyrrolo[1,2-c]imidazole-3-thione, and
(R)-1-(3-(pyrrolidin-1-yl)propyl)-6-(2,3,6-trifluorophenyl)-2,5,6,7-tetrahydro-3H-pyrrolo[1,2-c]imidazole-3-thione hydrofluoride
   are excluded.

This invention is also directed to a compound of formula I, as defined above, or a pharmaceutically acceptable salt or solvate thereof, for use in therapy.

This invention is also directed to a compound of formula I, as defined above, or a pharmaceutically acceptable salt or solvate thereof, for use in the treatment of conditions ameliorated by inhibition of DβH outside the CNS.

This invention is also directed to a compound of formula I, as defined above, or a pharmaceutically acceptable salt or solvate thereof, in the manufacture of a medicament for treatment of conditions ameliorated by inhibition of DβH outside the CNS.

This invention is also directed to a method for treating or preventing conditions ameliorated by inhibition of DβH outside the CNS comprising administering a therapeutically effective amount of a compound of formula I, as defined above, or a pharmaceutically acceptable salt or solvate thereof, to a patient in need thereof.

This invention is also directed to a pharmaceutical composition comprising (i) a therapeutically effective amount of a compound of formula I, as defined above, or a pharmaceutically acceptable salt or solvate thereof; and (ii) a pharmaceutically acceptable excipient.

Certain compounds of formula I may exist as tautomers. Where tautomers exist, each tautomeric form, and mixtures thereof, are contemplated as included in the present invention. Any reference in this specification to one specific tautomer of a compound of formula I is understood to encompass every tautomeric form as well as any mixtures thereof, in any ratio. The same applies to tautomers of more specific embodiments of compounds of formula I described herein, such as, but not limited to, tautomers of compounds of formula Ia, Ib, Ic, Id, Ie and If described below, and tautomers of the specific examples described in the experimental section below.

### DESCRIPTION OF THE FIGURE

FIG. 1 shows levels of noradrenaline (NA) in brain stem (Br.s) and heart left ventricle (Hrt.lv) at 15 h post-dose after oral administration of 10 mg/kg of compounds 1, 5, 6, 9, 11 and 14. Data are presented as % of Control. Each column represents mean±SEM of 4 to 5 rats per group.

### DETAILED DESCRIPTION OF THE EMBODIMENTS

### A. Definitions

“C₁-C₆ alkyl” means a monovalent unsubstituted saturated straight-chain or branched-chain hydrocarbon radical having from 1 to 6 carbon atoms. “C₁-C₂ alkyl”, “C₁-C₃ alkyl”, “C₁-C₄ alkyl” and “C₁-C₅ alkyl” have analogous meanings.

“partially or fully deuterated C₁-C₆ alkyl” means a C₁-C₆ alkyl wherein some or all of the hydrogen atoms have been replaced by deuterium.

“C₃-C₆ cycloalkyl” means a monovalent unsubstituted saturated cyclic hydrocarbon radical having from 3 to 6 carbon atoms.

“5- or 6-membered heterocyclyl” means a saturated monocyclic group with a total of 5 atoms in the ring wherein 1 or 2 of those atoms are each independently selected from N, O and S; or a saturated monocyclic group with a total of 6 atoms in the ring wherein 1 or 2 of those atoms are each independently selected from N, O and S. 5-membered heterocyclyl groups include pyrrolidinyl, tetrahydrofuranyl, tetrahydrothienyl (also called terahydrothiophenyl), imidazolidinyl, pyrazolidinyl, dioxolanyl, dithiolanyl, oxazolidinyl, isoxazolidinyl, thiazolidinyl and isothiazolidinyl. 6-membered heterocyclyl groups include piperidinyl, tetrahydropyranyl, tetrahydrothiopyranyl, piperazinyl, dioxanyl, dithianyl, morpholinyl and thiomorpholinyl.

“5- or 6-membered N-heterocyclyl” means a saturated monocyclic group with a total of 5 atoms in the ring wherein 1 of those atoms is N and another one of those atoms is optionally selected from N, O and S; or a saturated monocyclic group with a total of 6 atoms in the ring wherein 1 of those atoms is N and another one of those atoms is optionally independently selected from N, O and S. 5-membered N-heterocyclyl groups include pyrrolidinyl, imidazolidinyl, pyrazolidinyl, oxazolidinyl, isoxazolidinyl, thiazolidinyl and isothiazolidinyl. 6-membered N-heterocyclyl groups include piperidinyl, piperazinyl, morpholinyl and thiomorpholinyl.

“halo” means a fluorine (which may be depicted as —F), chlorine (which may be depicted as —Cl), bromine (which may be depicted as —Br) or iodine (which may be depicted as —I) radical.

“pharmaceutically acceptable salt” means a salt such as those described in standard texts on salt formation, see for example: P. Stahl, et al., Handbook of Pharmaceutical Salts: Properties, Selection and Use (VCHA/Wiley-VCH, 2002), or S. M. Berge, et al., “Pharmaceutical Salts” (1977) Journal of Pharmaceutical Sciences, 66, 1-19.

“pharmaceutically acceptable solvate” means a molecular complex comprising the compound of the invention and one or more pharmaceutically acceptable solvent molecules, for example, water or ethanol. The term “hydrate” maybe employed when said solvent is water. Pharmaceutically acceptable solvates include hydrates and other solvates wherein the solvent of crystallization may be isotopically substituted, e.g. D₂O, d₆-acetone, d₆-DMSO.

“pharmaceutically acceptable excipient” means any ingredient other than the compound(s) of the invention, or other known pharmacologically active components. The choice of excipient will to a large extent depend on factors such as the particular mode of administration, the effect of the excipient on solubility and stability, and the nature of the dosage form.

“therapy”, “treatment” and “treating” include both preventative and curative treatment of a condition, disease or disorder. It also includes slowing, interrupting, controlling or stopping the progression of a condition, disease or disorder. It also includes preventing, curing, slowing, interrupting, controlling or stopping the symptoms of a condition, disease or disorder.

Other variations to the disclosed embodiments can be understood and effected by those skilled in the art in practicing the claimed invention, from a study of the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage.

### B. Compounds

The invention provides a compound of formula I, as defined above, or a pharmaceutically acceptable salt or solvate thereof:

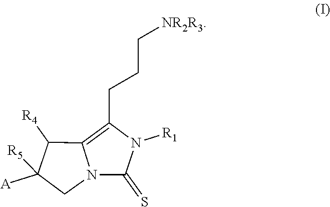

with the proviso that the compounds (R)-1-(3-(pyrrolidin-1-yl)propyl)-6-(2,3,5,6-tetrafluorophenyl)-2,5,6,7-tetrahydro-3H-pyrrolo[1,2-c]imidazole-3-thione hydrochloride, (R)-1-(3-(pyrrolidin-1-yl)propyl)-6-(2,3,5,6-tetrafluorophenyl)-2,5,6,7-tetrahydro-3H-pyrrolo[1,2-c]imidazole-3-thione, and (R)-1-(3-(pyrrolidin-1-yl)propyl)-6-(2,3,6-trifluorophenyl)-2,5,6,7-tetrahydro-3H-pyrrolo[1,2-c]imidazole-3-thione hydrofluoride are excluded.

### B0. Core Structures

In some embodiments of formula I, R₄ and R₅ combine, together with the carbon atom to which they are attached, to form a structure of formula Ia:

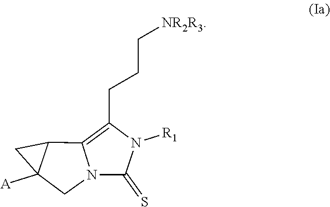

In some embodiments more than 50%, preferably more than 90%, more preferably more than 95% and even more preferably more than 99% of substituents R₅ and A of compounds of formula I have the stereochemical configuration of formula Ib

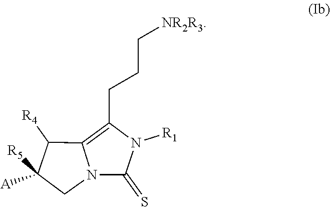

In some preferred embodiments of formula Ib, R₄ and R₅ combine, together with the carbon atoms to which they are attached, to form a cyclopropyl ring such that more than 50%, preferably more than 90%, more preferably more than 95% and even more preferably more than 99% of substituent A has the stereochemical configuration of formula Ic

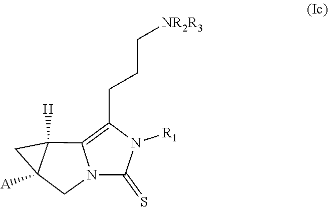

In some embodiments more than 50%, preferably more than 90%, more preferably more than 95% and even more preferably more than 99% of substituents R₅ and A of compounds of formula I have the stereochemical configuration of formula Id

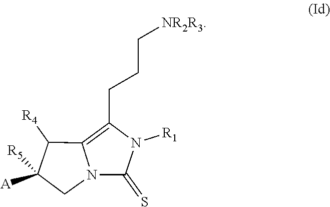

Preferred embodiments of formula I include compounds of formula Ie

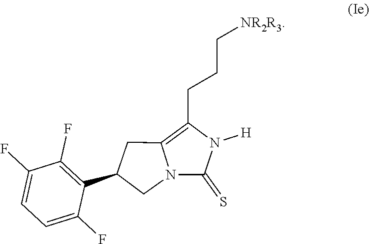

Other preferred embodiments of formula I include compounds of formula If

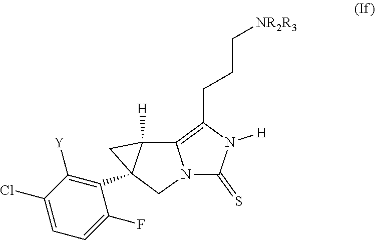

wherein Y is hydrogen or fluoro.

### B1. Substituent R₁

R₁ is hydrogen.

### B2. Substituents R₂ and R₃

R₂ is hydrogen; and

R₃ is hydrogen, methyl, 6-membered heterocyclyl, or CH₂X wherein X is a 5- or 6-membered heterocyclyl; or

R₂ is methyl; and

R₃ is methyl, 5- or 6-membered heterocyclyl, or CH₂X wherein X is 5- or 6-membered heterocyclyl; or

R₂ and R₃ combine, together with the N atom to which they are attached, to form a 5- or 6-membered N-heterocyclyl optionally substituted with one fluoro substituent.

In some embodiments R₂ is hydrogen and R₃ is hydrogen, methyl, 6-membered heterocyclyl, or CH₂X wherein X is 6-membered heterocyclyl.

In some embodiments R₂ is methyl and R₃ is methyl, 5- or 6-membered heterocycle, or CH₂X wherein X is 6-membered heterocyclyl.

In some embodiments R₂ and R₃ combine, together with the N atom to which they are attached, to form a 5- or 6-membered N-heterocyclyl optionally substituted with one fluoro substituent.

In some preferred embodiments R₂ is hydrogen and R₃ is hydrogen, methyl, tetrahydropyranyl, or CH₂X wherein X is tetrahydropyranyl.

In some preferred embodiments R₂ is methyl and R₃ is methyl, tetrahydrofuranyl, tetrahydropyranyl, or CH₂X wherein X is tetrahydropyranyl.

In some preferred embodiments R₂ and R₃ combine together with the N atom to which they are attached to form a pyrrolidinyl, 3-fluoropyrrolidinyl, piperidinyl or morpholinyl group.

### B3. Substituent R₄ (when not Combined with R₅)

R₄ is hydrogen.

### B4. Substituent R₅ (when not Combined with R₄)

R₅ is hydrogen.

### B5. Substituent A

A is

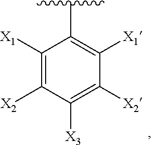

wherein:
X₁ is hydrogen or halo;
X₁′ is hydrogen or halo;
X₂ is hydrogen or halo;
X₂′ is hydrogen or halo; and
X₃ is hydrogen.

Preferably A is

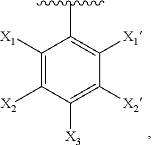

wherein:
X₁ is hydrogen, fluoro or chloro;
X₁′ is hydrogen, fluoro or chloro;
X₂ is hydrogen, fluoro, chloro or bromo;
X₂′ is hydrogen, fluoro, chloro or bromo; and
X₃ is hydrogen.

More preferably A is

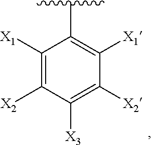

wherein:
X₁ is hydrogen or fluoro;
X₁′ is fluoro;
X₂ is fluoro or chloro;
X₂′ is hydrogen; and
X₃ is hydrogen.

In one preferred embodiment not all of X₁, X₁′, X₂, X₂′ and X₃ are hydrogen. Preferably A is selected from the group consisting of

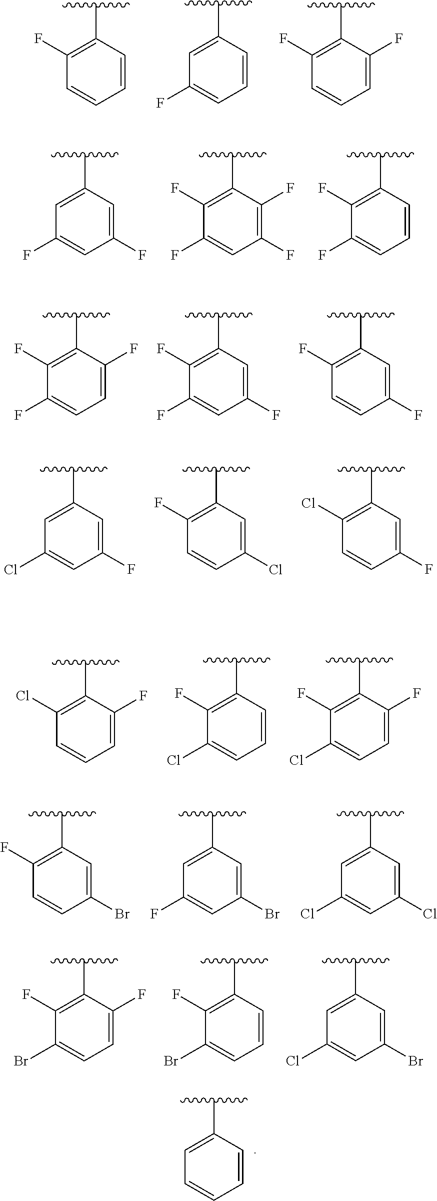

More preferably A is selected from the group consisting of

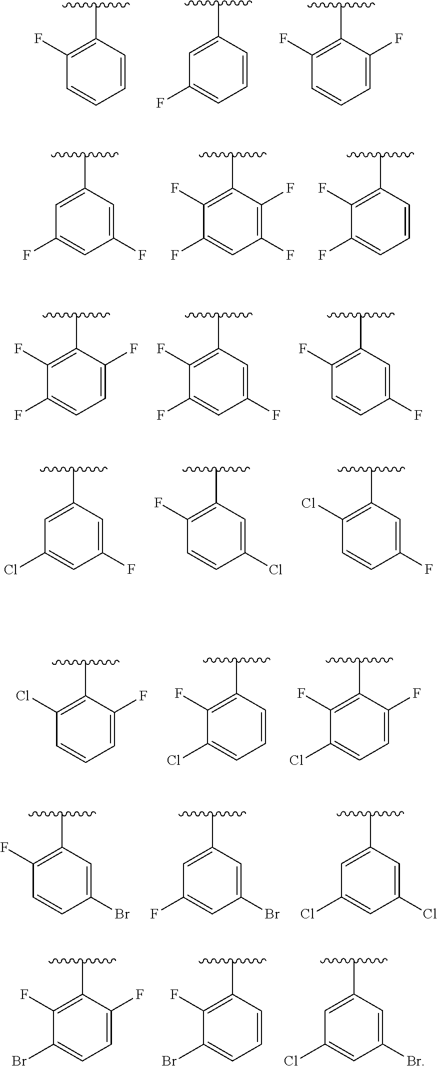

Even more preferably A is selected from the group consisting of

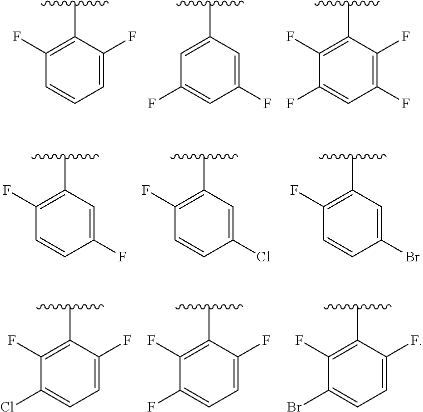

Most preferably A is selected from the group consisting of

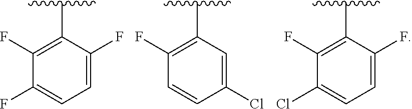

### B6. Specific Embodiments of Compounds of Formula I

Various embodiments of substituents R₁, R₂, R₃, R₄, R₅, A, X, X₁, X₁′, X₂, X₂′ and X₃ have been discussed in B1 to B5 above. These “substituent” embodiments can be combined with any of the “core structure” embodiments, discussed in B0 above, to form further embodiments of compounds of formula I. All embodiments of compounds of formula I formed by combining the “substituent” embodiments and “core structure” embodiments, discussed above, are within the scope of Applicants' invention, and some preferred further embodiments of the compounds of formula I are provided below.

In some embodiments of formula I a structure of formula Ie is highly preferred

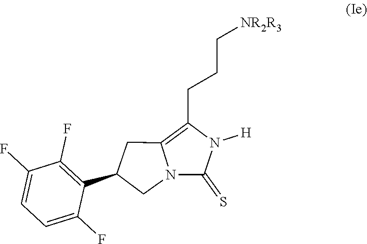

wherein:
R₂ is hydrogen; and
R₃ is hydrogen, methyl, 6-membered heterocyclyl, or CH₂X wherein X is a 5- or 6-membered heterocyclyl; or
R₂ is methyl; and
R₃ is methyl, 5- or 6-membered heterocyclyl, or CH₂X wherein X is a 5- or 6-membered heterocyclyl; or
R₂ and R₃ combine together with the N atom to which they are attached to form a 5- or 6-membered N-heterocyclyl optionally substituted with one fluoro substituent.
   Preferably wherein:
R₂ is hydrogen; and
R₃ is hydrogen, methyl, tetrahydropyranyl, or CH₂X wherein X is tetrahydrofuranyl or tetraydropyranyl; or
R₂ is methyl; and
R₃ is methyl, tetrahydrofuranyl, tetrahydropyranyl, or CH₂X wherein X is tetrahydrofuranyl or tetraydropyranyl; or
R₂ and R₃ combine together with the N atom to which they are attached to form a 3-fluoropyrrolidinyl or morpholinyl group.

In some embodiments of formula I a structure of formula If is highly preferred

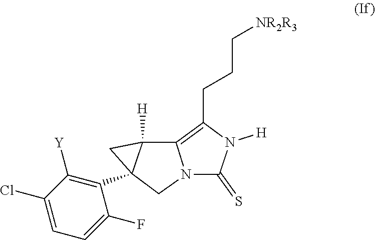

wherein:
R₂ is hydrogen; and
R₃ is 6-membered heterocyclyl; or
R₂ and R₃ combine together with the N atom to which they are attached to form a 5- or 6-membered N-heterocyclyl; and
Y is hydrogen or fluoro.
   Preferably wherein:
R₂ is hydrogen; and
R₃ is tetrahydropyranyl; or
R₂ and R₃ combine together with the N atom to which they are attached to form a pyrrolidinyl or morpholinyl.

Especially preferred embodiments of compounds of formula I are described in Examples 1 to 22 below. Where these examples describe the preparation of a compound of formula I in the form of a pharmaceutically acceptable salt or solvate, it will be appreciated that the present invention also relates to said compound in the form of the corresponding free acid or free base. Similarly, where these examples describe the preparation of a compound of formula I in the form of a free acid or free base, it will be appreciated that the present invention also relates to said compound in the form of a pharmaceutically acceptable salt or solvate thereof.

The non-salt, non-solvated forms of Examples 1 to 22 are listed below. The invention also relates to the pharmaceutically acceptable salts or solvates of each of these individual compounds. Should any of these compounds exist as tautomers, each tautomeric form, and mixtures thereof, are contemplated as included in the present invention.
Example 1: (R)-1-(3-morpholinopropyl)-6-(2,3,6-trifluorophenyl)-2,5,6,7-tetrahydro-3H-pyrrolo[1,2-c]imidazole-3-thione
Example 2: (R)-1-(3-aminopropyl)-6-(2,3,6-trifluorophenyl)-2,5,6,7-tetrahydro-3H-pyrrolo[1,2-c]imidazole-3-thione
Example 3: (R)-1-(3-(((tetrahydro-2H-pyran-4-yl)methyl)amino)propyl)-6-(2,3,6-trifluorophenyl)-2,5,6,7-tetrahydro-3H-pyrrolo[1,2-c]imidazole-3-thione
Example 4: (R)-1-(3-aminopropyl)-6-(2,3,6-trifluorophenyl)-2,5,6,7-tetrahydro-3H-pyrrolo[1,2-c]imidazole-3-thione
Example 5: (R)-1-(3-((tetrahydro-2H-pyran-4-yl)amino)propyl)-6-(2,3,6-trifluorophenyl)-2,5,6,7-tetrahydro-3H-pyrrolo[1,2-c]imidazole-3-thione
Example 6: (R)-1-(3-morpholinopropyl)-6-(2,3,6-trifluorophenyl)-2,5,6,7-tetrahydro-3H-pyrrolo[1,2-c]imidazole-3-thione
Example 7: (R)-1-(3-((R)-3-fluoropyrrolidin-1-yl)propyl)-6-(2,3,6-trifluorophenyl)-2,5,6,7-tetrahydro-3H-pyrrolo[1,2-c]imidazole-3-thione
Example 8: (R)-1-(3-(dimethylamino)propyl)-6-(2,3,6-trifluorophenyl)-2,5,6,7-tetrahydro-3H-pyrrolo[1,2-c]imidazole-3-thione
Example 9: (R)-1-(3-(((S)-tetrahydro-2H-pyran-3-yl)amino)propyl)-6-(2,3,6-trifluorophenyl)-2,5,6,7-tetrahydro-3H-pyrrolo[1,2-c]imidazole-3-thione
Example 10: (R)-1-(3-(dimethylamino)propyl)-6-(2,3,6-trifluorophenyl)-2,5,6,7-tetrahydro-3H-pyrrolo[1,2-c]imidazole-3-thione
Example 11: (R)-1-(3-((R)-3-fluoropyrrolidin-1-yl)propyl)-6-(2,3,6-trifluorophenyl)-2,5,6,7-tetrahydro-3H-pyrrolo[1,2-c]imidazole-3-thione
Example 12: (R)-1-(3-(methyl(tetrahydro-2H-pyran-4-yl)amino)propyl)-6-(2,3,6-trifluorophenyl)-2,5,6,7-tetrahydro-3H-pyrrolo[1,2-c]imidazole-3-thione
Example 13: (6R)-1-(3-(methyl(tetrahydrofuran-3-yl)amino)propyl)-6-(2,3,6-trifluorophenyl)-2,5,6,7-tetrahydro-3H-pyrrolo[1,2-c]imidazole-3-thione
Example 14: (R)-1-(3-(methyl(tetrahydro-2H-pyran-4-yl)amino)propyl)-6-(2,3,6-trifluorophenyl)-2,5,6,7-tetrahydro-3H-pyrrolo[1,2-c]imidazole-3-thione
Example 15: (5aS,6aR)-5a-(3-chloro-2,6-difluorophenyl)-1-(3-morpholinopropyl)-5,5a,6,6a-tetrahydrocyclopropa[3,4]pyrrolo[1,2-c]imidazole-3(2H)-thione
Example 16: (5aS,6aR)-5a-(5-chloro-2-fluorophenyl)-1-(3-morpholinopropyl)-5,5a,6,6a-tetrahydrocyclopropa[3,4]pyrrolo[1,2-c]imidazole-3(2H)-thione
Example 17: (5aS,6aR)-5a-(5-chloro-2-fluorophenyl)-1-(3-((tetrahydro-2H-pyran-4-yl)amino)propyl)-5,5a,6,6a-tetrahydrocyclopropa[3,4]pyrrolo[1,2-c]imidazole-3(2H)-thione
Example 18: (5aS,6aR)-5a-(3-chloro-2,6-difluorophenyl)-1-(3-((tetrahydro-2H-pyran-4-yl)amino)propyl)-5,5a,6,6a-tetrahydrocyclopropa[3,4]pyrrolo[1,2-c]imidazole-3(2H)-thione
Example 19: (5aS,6aR)-5a-(3-chloro-2,6-difluorophenyl)-1-(3-(pyrrolidin-1-yl)propyl)-5,5a,6,6a-tetrahydrocyclopropa[3,4]pyrrolo[1,2-c]imidazole-3(2H)-thione
Example 20: (5aS,6aR)-5a-(5-chloro-2-fluorophenyl)-1-(3-(pyrrolidin-1-yl)propyl)-5,5a,6,6a-tetrahydrocyclopropa[3,4]pyrrolo[1,2-c]imidazole-3(2H)-thione
Example 21: (5aS,6aR)-5a-(5-chloro-2-fluorophenyl)-1-(3-(((S)-tetrahydro-2H-pyran-3-yl)amino)propyl)-5,5a,6,6a-tetrahydrocyclopropa[3,4]pyrrolo[1,2-c]imidazole-3(2H)-thione
Example 22: (5aS,6aR)-5a-(3-chloro-2,6-difluorophenyl)-1-(3-(((S)-tetrahydro-2H-pyran-3-yl)amino)propyl)-5,5a,6,6a-tetrahydrocyclopropa[3,4]pyrrolo[1,2-c]imidazole-3(2H)-thione

### C. Compositions

The compounds of the invention intended for pharmaceutical use may be administered alone or in combination with one or more other compounds of the invention or in combination with one or more other drugs (or as any combination thereof). Generally, they will be administered as a formulation in association with one or more pharmaceutically acceptable excipients. Accordingly, the present invention is also directed to a pharmaceutical composition comprising (i) a therapeutically effective amount of a compound of formula I, as defined above, or a pharmaceutically acceptable salt or solvate thereof, and (ii) a pharmaceutically acceptable excipient.

Pharmaceutical compositions suitable for the delivery of compounds of the present invention and methods for their preparation will be readily apparent to those skilled in the art. Such compositions and methods for their preparation may be found, for example, in “Remington's Pharmaceutical Sciences”, 19th Edition (Mack Publishing Company, 1995).

### D. Methods of Use

This invention is also directed to compounds of formula I, as defined above, or a pharmaceutically acceptable salt or solvate thereof, for use in therapy, in particular for the treatment of conditions ameliorated by inhibition of DβH outside the CNS.

This invention is also directed to the use of compounds of formula I, as defined above, or a pharmaceutically acceptable salt or solvate thereof, in the manufacture of a medicament for treatment of conditions ameliorated by inhibition of DβH outside the CNS.

This invention is also directed to a method for treating conditions ameliorated by inhibition of dopamine-beta-hydroxylase outside the CNS comprising administering a therapeutically effective amount of a compound of formula I, as defined in claim 1, or a pharmaceutically acceptable salt or solvate thereof, to a patient in need thereof.

Conditions ameliorated by inhibition of DβH outside the CNS can include, but are not limited to: cardiovascular disorders such as Angina, Hypertension, Chronic or Congestive Heart Failure, Pulmonary Hypertension (PH) and Pulmonary Arterial Hypertension (PAH).

Reference is made to the “Guidelines for the diagnosis and treatment of pulmonary hypertension” (European Heart Journal (2009) 30, 2493-2537) for details on the definition, classification and pathology and pathobiological features of PH.

Typically, pulmonary hypertension is a group of diseases characterized by a progressive increase of pulmonary vascular resistance leading to right ventricular failure and premature death. It may be defined by a mean pulmonary artery pressure equal or greater than 25 mmHg at rest.

PH has been clinically classified by the WHO into 5 groups, according to the cause of the disease, and symptoms may differ, depending on the ‘group’ that caused the disease.

However, ‘common’ symptoms are as follows:
Difficulty in breathing or shortness of breath (main symptom)
Fatigue
Dizziness
Swelling in the ankles or legs (edema)
Bluish lips and skin (cyanosis)
Chest pain
Racing pulse and palpitations

A clinical classification of pulmonary hypertension (PH) has been undertaken and reported by McLaughlin et al in “ACCF/AHA 2009 Expert Consensus Document on Pulmonary Hypertension”, J Am Coll Cardiol 53, 1573-1619, 2009. PH was classified as follows:
1. Pulmonary arterial hypertension (PAH)
   1.1. Idiopathic (IPAH)
   1.2. Familial (FPAH)
   1.3. Associated with (APAH):
      1.3.1. Connective tissue disorder
      1.3.2. Congenital systemic-to-pulmonary shunts
      1.3.3. Portal hypertension
      1.3.4. HIV infection
      1.3.5. Drugs and toxins
      1.3.6. Other (thyroid disorders, glycogen storage disease, Gaucher's disease, hereditary hemorrhagic telangiectasia, hemoglobinopathies, chronic myeloproliferative disorders, splenectomy)
   1.4. Associated with significant venous or capillary involvement
      1.4.1. Pulmonary veno-occlusive disease (PVOD)
      1.4.2. Pulmonary capillary hemangiomatosis (PCH)
   1.5. Persistent pulmonary hypertension of the newborn
2. Pulmonary hypertension with left heart disease
   2.1. Left-sided atrial or ventricular heart disease
   2.2. Left-sided valvular heart disease
3. Pulmonary hypertension associated with lung diseases and/or hypoxemia
   3.1. Chronic obstructive pulmonary disease
   3.2. Interstitial lung disease
   3.3. Sleep disordered breathing
   3.4. Alveolar hypoventilation disorders
   3.5. Chronic exposure to high altitude
   3.6. Developmental abnormalities
4. Pulmonary hypertension due to chronic thrombotic and/or embolic disease (CTEPH)
   4.1. Thromboembolic obstruction of proximal pulmonary arteries
   4.2. Thromboembolic obstruction of distal pulmonary arteries
   4.3. Nonthrombotic pulmonary embolism (tumor, parasites, foreign material)
5. Miscellaneous

Sarcoidosis, histiocytosis X, lymphangiomatosis, compression of pulmonary vessels (adenopathy, tumor, fibrosing mediastinitis)

The WHO has also provided the following functional assessment classification:

Functional Symptomatic profile

Class
I Patients with pulmonary hypertension but without resulting limitation of physical activity. Ordinary physical activity does not cause dyspnoea or fatigue, chest pain, or near syncope
II Patients with pulmonary hypertension resulting in slight limitation of physical activity. They are comfortable at rest. Ordinary physical activity causes undue dyspnoea or fatigue, chest pain, or near syncope
III Patients with pulmonary hypertension resulting in marked limitation of physical activity. They are comfortable at rest. Less than ordinary activity causes undue dyspnoea or fatigue, chest pain, or near syncope
IV Patients with pulmonary hypertension with inability to carry out any physical activity without symptoms. These patients manifest signs of right heart failure. Dyspnoea and/or fatigue may even be present at rest. Discomfort is increased by any physical activity.

### E. General Synthetic Methodology

The methods used for the synthesis of the compounds of the invention are illustrated by the schemes below. The starting materials and reagents used in preparing these compounds are available from commercial suppliers or can be prepared by methods obvious to those skilled in the art.

The starting material for compounds of formula Ie (hereafter referred to as intermediate 1) can generally be synthesised by the method outlined in Scheme 1 as either enriched enantiomers or racemates:

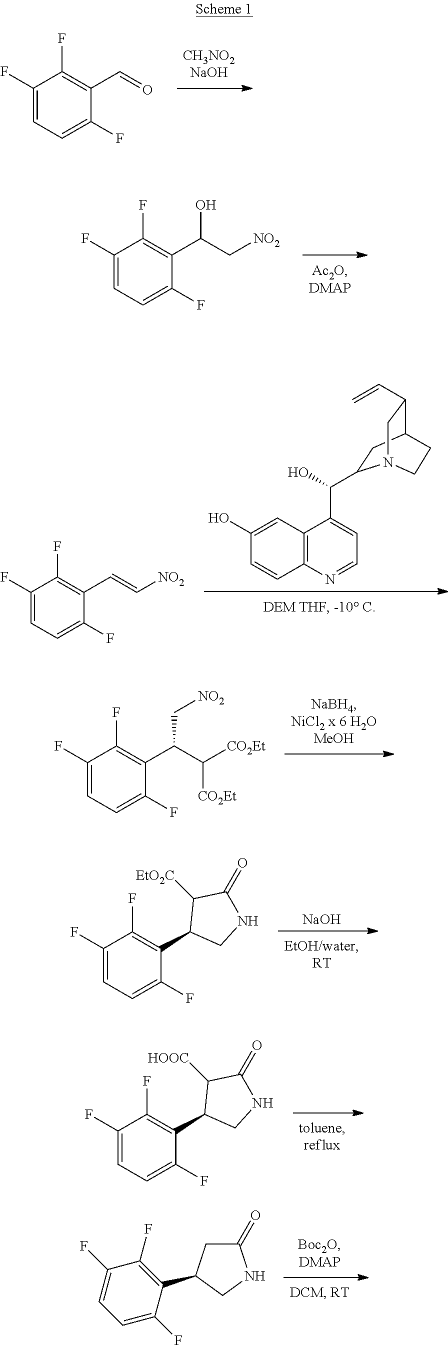

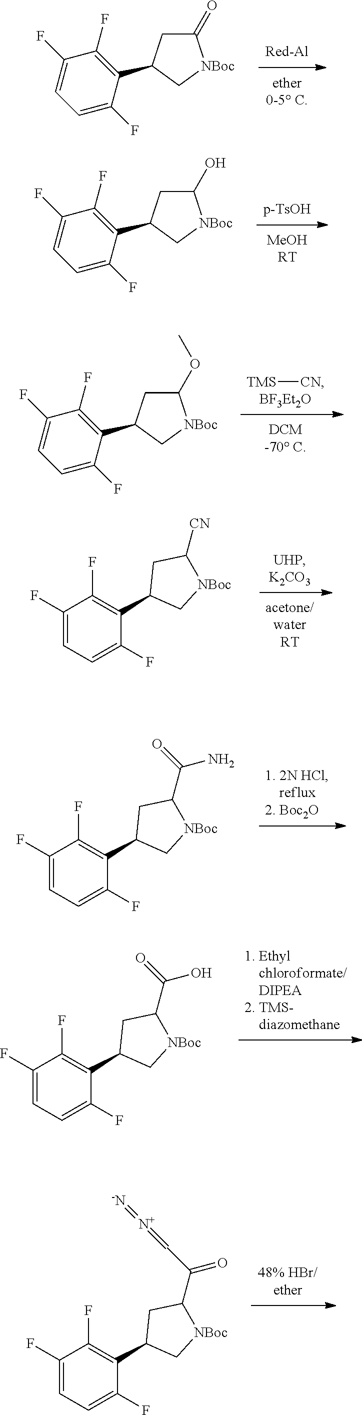

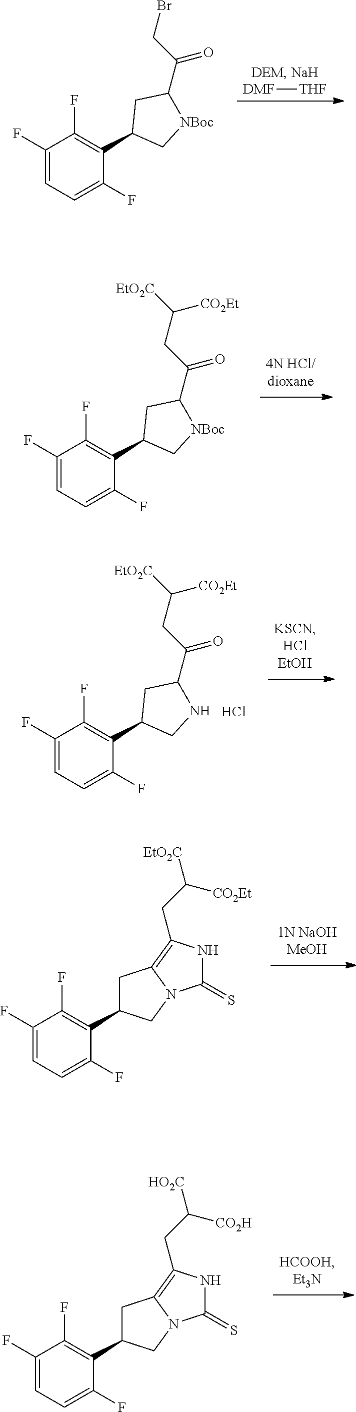

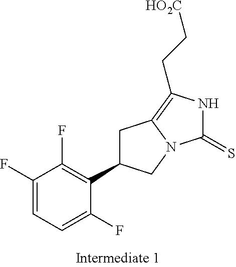 [SMILES: O=C(O)CCc1[nH]c(=S)n2c1C[C@H](c1c(F)ccc(F)c1F)C2]

The starting materials for compounds of formula If (hereafter referred to as intermediates 2 and 3) can generally be synthesised by the method outlined in Scheme 2 as either enriched enantiomers or racemates:

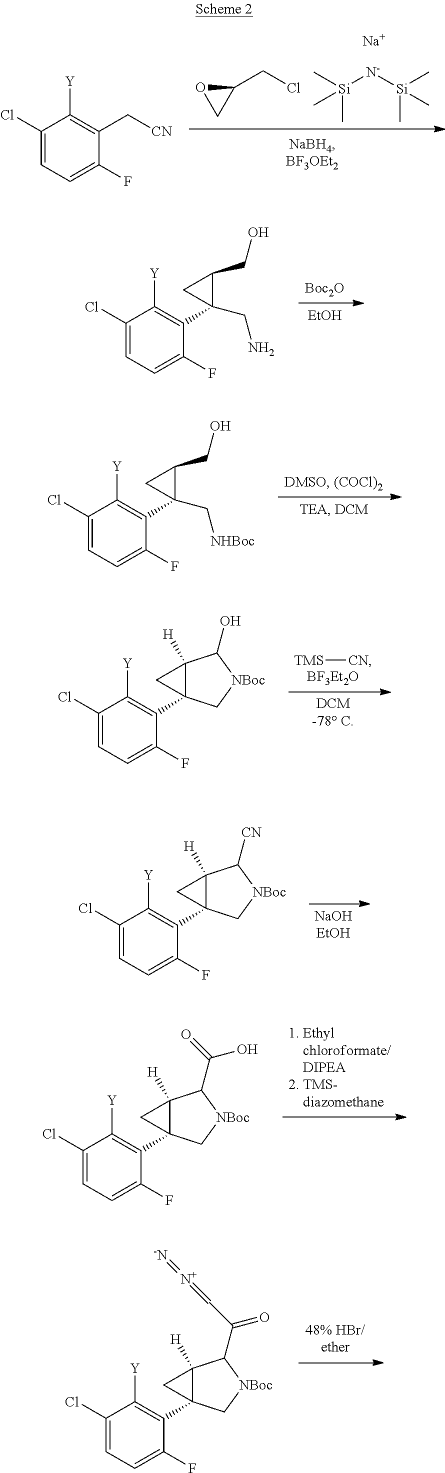

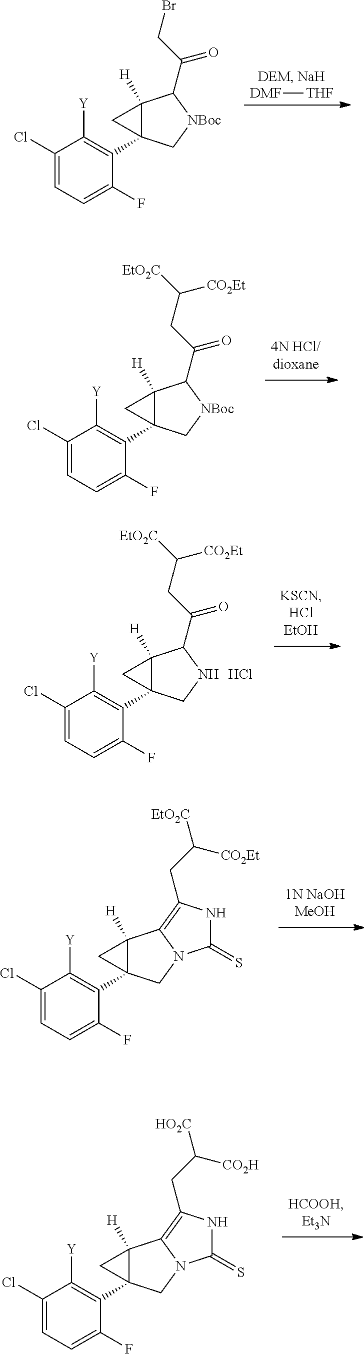

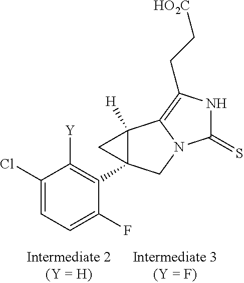

Compounds of formula Ie or If, with various identities for R₂ and R₃, can generally be synthesised by the methods outlined in Scheme 3 as either enriched enantiomers or racemates:

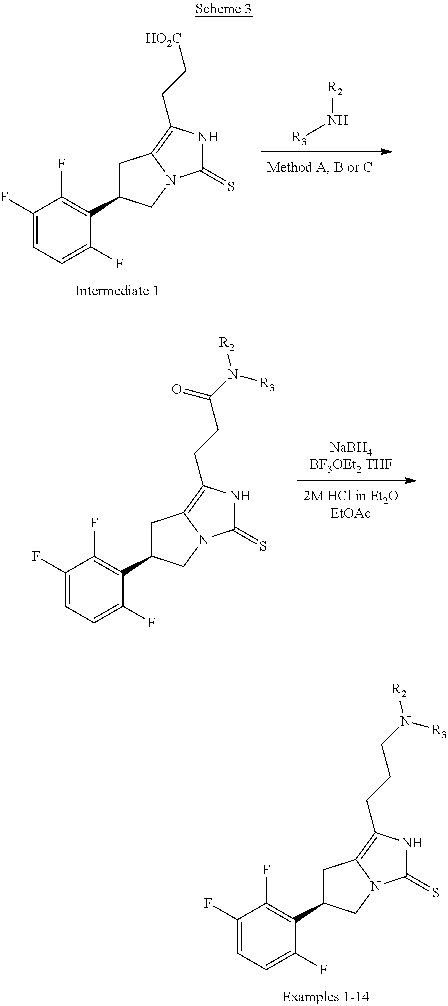

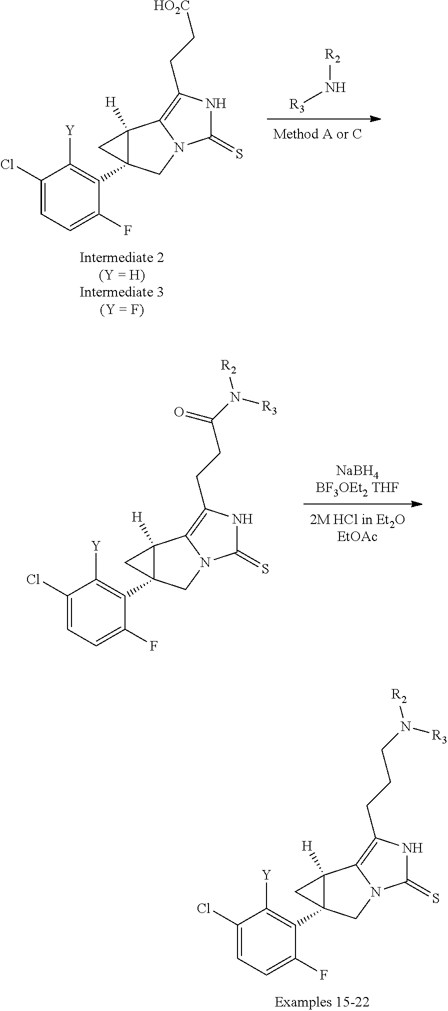

### F. Examples

All compounds and intermediates were characterised by NMR. The spectra were recorded on a Bruker Avance III 600 MHz spectrometer with solvent used as internal standard. ¹³C spectra were recorded at 150 MHz and ¹H spectra were recorded at 600 MHz. Data are reported in the following order: approximate chemical shift (ppm), number of protons, multiplicity (br, broad; d, doublet; m, multiplet; s, singlet; t, triplet) and coupling constant (Hz).

Room temperature in the following protocols means the temperature ranging from 20° C. to 25° C.

### Intermediate 1: (R)-3-(3-thioxo-6-(2,3,6-trifluorophenyl)-2,5,6,7-tetrahydro-3H-pyrrolo[1,2-c]imidazol-1-yl)propanoic acid

### Step 1: 2-nitro-1-(2,3,6-trifluorophenyl)ethan-1-ol

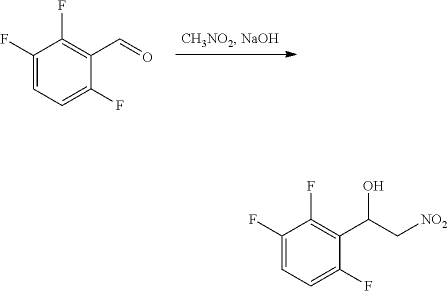 [SMILES: O=Cc1c(F)ccc(F)c1F ; O=[N+]([O-])CC(O)c1c(F)ccc(F)c1F]

To a solution of methanol (60 mL), water (30 mL), and 2.5 M sodium hydroxide (28.7 mL, 71.8 mmol) was added a solution of 2,3,6-trifluorobenzaldehyde (10 g, 62.5 mmol) and nitromethane (3.87 mL, 71.8 mmol) in methanol (10 mL) dropwise over 30 min at 5° C., while the internal temperature was maintained between 5 and 10° C. with external cooling. The reaction was then agitated in the cold for an additional 0.5 h, and then a solution of cc. HCl (10.41 mL, 125 mmol) in water (30 mL) was added in one portion at 0-10° C. with stirring. The mixture was extracted with dichloromethane (ca. 200 mL), the organic phase was washed with brine, dried over MgSO4, filtered and evaporated to dryness to give the title product (Yield: 13.05 g, 94%).

### Step 2: (E)-1,2,4-trifluoro-3-(2-nitrovinyl)benzene

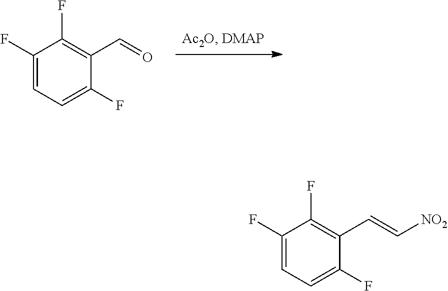 [SMILES: O=Cc1c(F)ccc(F)c1F ; O=[N+]([O-])/C=C/c1c(F)ccc(F)c1F]

To a solution of 2-nitro-1-(2,3,6-trifluorophenyl)ethanol (13.04 g, 59.0 mmol) and N,N-dimethylpyridin-4-amine (0.720 g, 5.90 mmol) in dichloromethane (120 mL) was added acetic anhydride (6.68 mL, 70.8 mmol) at ambient temperature and the mixture was stirred for 16 h. Thereupon, it was washed with water and sodium bicarbonate, respectively. After being dried over MgSO₄, filtered through a short silica pad and evaporated to dryness to give (E)-1,2,4-trifluoro-3-(2-nitrovinyl)benzene as a light yellow powder (Yield: 11.55 g, 96%).

### Step 3: diethyl (R)-2-(2-nitro-1-(2,3,6-trifluorophenyl)ethyl)malonate

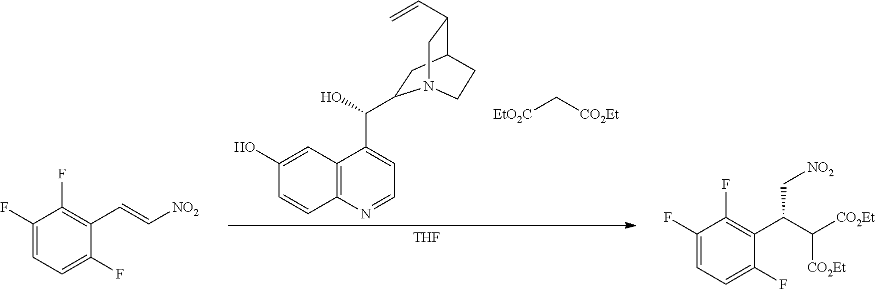

To a stirred solution of (E)-1,2,4-trifluoro-3-(2-nitrovinyl)benzene (5.7 g, 28.1 mmol) in dry tetrahydrofuran (40 mL) was added 4-((1S)-hydroxy((1S,4S,5R)-5-vinylquinuclidin-2-yl)methyl)quinolin-6-ol (0.218 g, 0.702 mmol) at room temperature with stirring followed by addition of diethyl malonate (5.56 mL, 36.5 mmol). The mixture was cooled to −5 to −10° C. under inert atmosphere and stirred for 16 h in the cold. Thereupon, the mixture was evaporated to dryness under vacuum and the residue was taken up in dichloromethane, washed with 1 M HCl (ca. 15 mL) and brine, respectively. After being dried over MgSO₄, the mixture was filtered and evaporated to give the title compound as a yellowish oil. Yield: 11.37 g, 95%).

### Step 4: ethyl (4R)-2-oxo-4-(2,3,6-trifluorophenyl)pyrrolidine-3-carboxylate

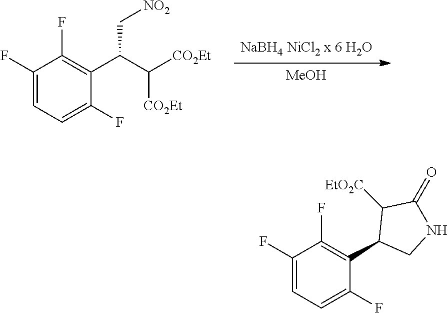 [SMILES: "MeOH" ; CCOC(=O)C(C(=O)OCC)[C@@H](C[N+](=O)[O-])c1c(F)ccc(F)c1F ; CCOC(=O)C1C(=O)NC[C@H]1c1c(F)ccc(F)c1F]

To a suspension of diethyl (R)-2-(2-nitro-1-(2,3,6-trifluorophenyl)ethyl)malonate (11.36 g, 26.6 mmol) in methanol (150 mL) was added nickel(II) chloride hexahydrate (6.32 g, 26.6 mmol) followed by addition of sodium borohydride (8.04 g, 213 mmol) in portions with ice cooling. The mixture was stirred for 5 h at room temperature, then quenched with 1.5 N HCl solution to pH=3. The mixture was stirred at ambient temperature for 16 h, which then was extracted with dichloromethane (150+75 mL), the organic phase was dried over MgSO₄ and evaporated to dryness. The obtained crude product was crystallized from petroleum ether to give beige powder. (Yield: 6.93 g, 91%).

### Step 5: (4R)-4-(2,3,6-trifluorophenyl)-2-oxopyrrolidine-3-carboxylic acid

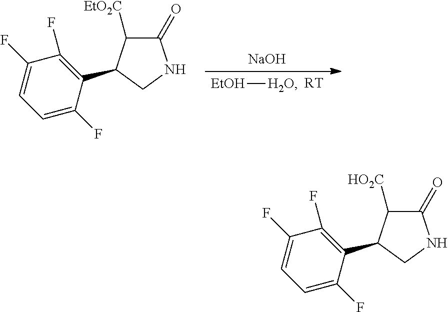

To a stirred solution of (4R)-ethyl 4-(2,3,6-trifluorophenyl)-2-oxopyrrolidine-3-carboxylate (6.92 g, 24.09 mmol) in ethanol (100 mL) was added 1 M sodium hydroxide (28.9 mL, 28.9 mmol). The resulting suspension was stirred for 2 h at room temperature, the organics were then removed under vacuum, and the residue was dissolved in water (ca. 100 mL), and then acidified by adding cc HCl (5.94 mL, 72.3 mmol). The mixture was aged in the cold, and then the resulting crystals were collected by filtration, washed with water and the dried under vacuum to give the titled product. Yield: 5.61 g, 90%.

### Step 6: (R)-4-(2,3,6-trifluorophenyl)pyrrolidin-2-one

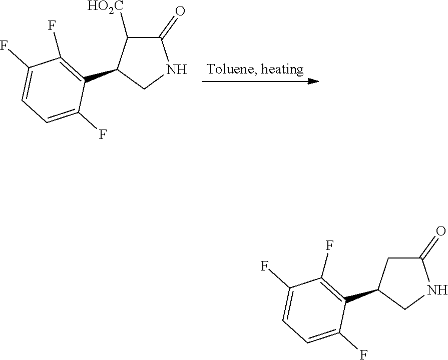 [SMILES: O=C(O)C1C(=O)NC[C@H]1c1c(F)ccc(F)c1F ; O=C1C[C@H](c2c(F)ccc(F)c2F)CN1]

A solution of (4R)-4-(2,3,6-trifluorophenyl)-2-oxopyrrolidine-3-carboxylic acid (5.6 g, 21.61 mmol) in toluene (200 mL) was stirred under reflux for 3 h, whereupon the mixture was evaporated to ca. 50 mL, and then diluted with petroleum ether (ca. 30 mL). The resulting crystals were collected, washed with petroleum ether and dried under vacuum to give a beige powder. Yield: 4.33 g, 93%.

### Step 7: (R)-tert-butyl 4-(2,3,6-trifluorophenyl)-2-oxopyrrolidine-1-carboxylate

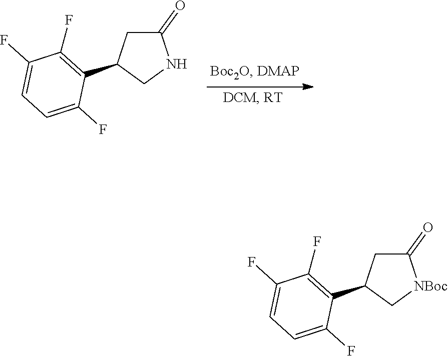 [SMILES: O=C1C[C@H](c2c(F)ccc(F)c2F)CN1 ; CC(C)(C)OC(=O)N1C[C@@H](c2c(F)ccc(F)c2F)CC1=O]

To a stirred solution of (R)-4-(2,3,6-trifluorophenyl)pyrrolidin-2-one (4.32 g, 20.08 mmol) in dry dichloromethane (16 mL) was added at room temperature di-tert-butyl dicarbonate (6.57 g, 30.1 mmol) followed by addition of N,N-dimethylpyridin-4-amine (2.453 g, 20.08 mmol). The mixture was then stirred at room temperature for 24 h, and then diluted with dichloromethane (100 mL). The mixture was washed with citric acid, dried over MgSO₄, filtered and then evaporated to dryness. Chromatography (petroleum ether-ethyl acetate) gave an oil which was crystallized from petroleum ether. The product was isolated as a white powder. Yield: 5.35 g, 85%.

### Step 8: (4R)-tert-butyl 4-(2,3,6-trifluorophenyl)-2-hydroxypyrrolidine-1-carboxylate

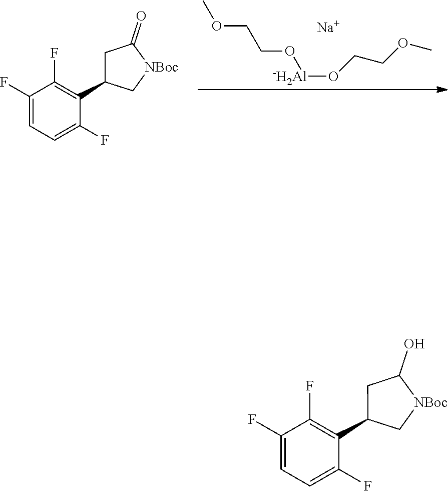

To a stirred solution of (R)-tert-butyl 4-(2,3,6-trifluorophenyl)-2-oxopyrrolidine-1-carboxylate (5.34 g, 16.94 mmol) in dry diethyl ether (51 mL) was added dropwise 65% RED-Al (bis(2-methoxyethoxy)aluminum(III) sodium hydride) (3.30 mL, 11.01 mmol) in toluene at 0-5° C. under nitrogen and the mixture was stirred for 0.5 h in the cold. Thereupon, the mixture was quenched with sodium bicarbonate solution and stirred for 30 min, the organic phase was separated and the aqueous phase was extracted with diethyl ether. The combined organic phases were dried over MgSO₄, filtered and then evaporated to dryness to give the product as a yellowish oil. (Yield: 6.08 g, 96%).

### Step 9: tert-butyl (4R)-2-methoxy-4-(2,3,6-trifluorophenyl)pyrrolidine-1-carboxylate

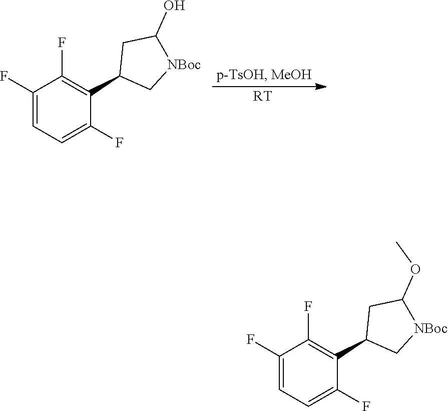 [SMILES: CC(C)(C)OC(=O)N1C[C@@H](c2c(F)ccc(F)c2F)CC1O ; "RT" ; COC1C[C@H](c2c(F)ccc(F)c2F)CN1C(=O)OC(C)(C)C]

To a stirred of (4R)-tert-butyl 2-hydroxy-4-(2,3,6-trifluorophenyl)pyrrolidine-1-carboxylate (6 g, 16.07 mmol) in methanol (160 mL) was added p-toluenesulfonic acid monohydrate (0.306 g, 1.607 mmol) at 20-25° C. and the solution was stirred for 24 h. Thereupon, the solution was neutralised by addition of 1M NaOH (1.607 mL, 1.607 mmol), and then stripped down to dryness. The residue was taken up in a mixture of ethyl acetate-petroleum ether (1:1), dried over MgSO₄, filtered through silica, and then evaporated to dryness to give (4R)-tert-butyl 2-methoxy-4-(2,3,6-trifluorophenyl)pyrrolidine-1-carboxylate (5.7 g, 96% yield) as a yellowish oil.

### Step 10: (4R)-tert-butyl 2-cyano-4-(2,3,6-trifluorophenyl)pyrrolidine-1-carboxylate

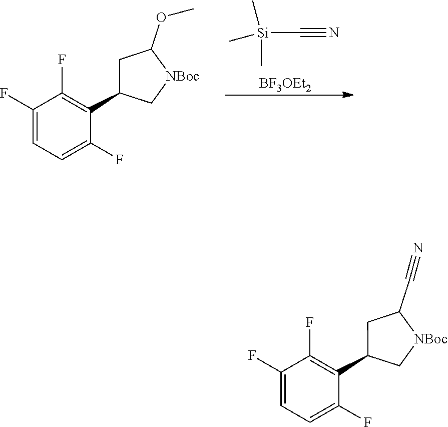

To a stirred solution of tert-butyl (4R)-2-methoxy-4-(2,3,6-trifluorophenyl)pyrrolidine-1-carboxylate (5.69 g, 15.46 mmol) in dry dichloromethane (110 mL) was added trimethylsilanecarbonitrile (4.14 mL, 30.9 mmol) followed by addition of boron trifluoride etherate (4.31 mL, 34.0 mmol) at −70° C. The mixture was stirred for 4 h in the cold, thereupon quenched with sodium bicarbonate solution, and then allowed to warm up with stirring to room temperature. The organic phase was dried over MgSO₄, filtered and evaporated to dryness under vacuum to give the title compound as a beige solid. (Yield: 5.78 g, 97%).

Step 11: tert-butyl (4R)-2-carbamoyl-4-(2,3,6-trifluorophenyl)pyrrolidine-1-carboxylate

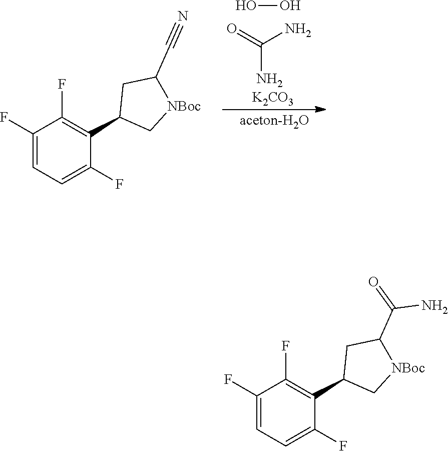

To a stirred solution of (4R)-tert-butyl 2-cyano-4-(2,3,6-trifluorophenyl)pyrrolidine-1-carboxylate (5.77 g, 15.03 mmol) in a mixture of acetone (75 mL) and water (25 mL) was added urea hydrogen peroxide complex (7.07 g, 75 mmol) followed by potassium carbonate (0.415 g, 3.01 mmol) and the solution was stirred at 20-25° C. for 16 h. Acetone was then partially removed on a rotavap until an oil separated. Thereupon, the mixture was diluted with water and petroleum ether, aged with stirring for 1 h (crystallisation occurred). The obtained solid collected, washed with water, petroleumether, and then dried to give (4R)-tert-butyl 2-carbamoyl-4-(2,3,6-trifluorophenyl)pyrrolidine-1-carboxylate as a white powder (4.43 g, 86% yield).

### Step 12: (4R)-1-(tert-butoxycarbonyl)-4-(2,3,6-trifluorophenyl)pyrrolidine-2-carboxylic acid

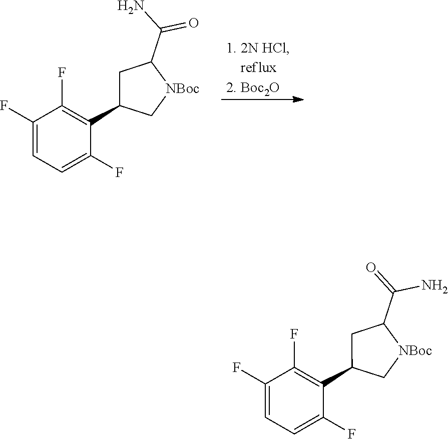

To a stirred suspension of (4R)-tert-butyl 2-carbamoyl-4-(2,3,6-trifluorophenyl)pyrrolidine-1-carboxylate (4.42 g, 12.84 mmol) in 2M HCl (96 mL, 193 mmol) was stirred under reflux for 16 h. Thereupon, the mixture was concentrated and the residue was dissolved in water. The mixture was then neutralized by addition of 1M NaOH (25.7 mL, 25.7 mmol) and the solution was concentrated to approx 50 mL. Methanol (70 mL) was added followed by addition of di-tert-butyl dicarbonate (3.08 g, 14.12 mmol) and the mixture was stirred for 45 min. Methanol was then removed under vacuum, the residue was diluted with water (50 mL) and washed with petroleum ether. The aqueous phase was acidified to pH=2 by addition of 2N HCl, and then extracted with dichloromethane. The organic phase was dried, stripped down to dryness to give (4R)-1-(tert-butoxycarbonyl)-4-(2,3,6-trifluorophenyl)pyrrolidine-2-carboxylic acid as a white powder (3.85 g, 87% yield).

### Step 13: tert-butyl (4R)-2-(2-diazoacetyl)-4-(2,3,6-trifluorophenyl)pyrrolidine-1-carboxylate

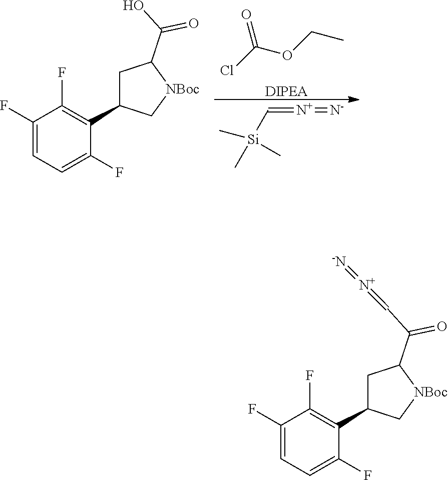

To a solution of (4R)-1-(tert-butoxycarbonyl)-4-(2,3,6-trifluorophenyl)pyrrolidine-2-carboxylic acid (9.22 g, 26.7 mmol) and N-ethyl-N-isopropylpropan-2-amine (8.16 mL, 46.7 mmol) in dry tetrahydrofuran (100 mL) was added ethyl chloroformate (3.85 mL, 40.1 mmol) at 0-5° C. The mixture was stirred for 4 h in the cold, and then diluted with acetonitrile (50 mL) followed by addition of 2 M (diazomethyl)trimethylsilane (26.7 mL 53.4 mmol) in diethyl ether. The stirring was continued for additional 3 h at 0-5° C. and the mixture was allowed to warm up naturally overnight with stirring under N₂. Thereupon, the solvents were removed under vacuum and the residue was purified by column chromatography in a mixture of petroleumether-ethyl acetate. to give (4R)-tert-butyl 2-(2-diazoacetyl)-4-(2,3,6-trifluorophenyl)pyrrolidine-1-carboxylate as a yellow oil. Yield: 6.92 g, 42%.

### Step 14: tert-butyl (4R)-2-(2-bromoacetyl)-4-(2,3,6-trifluorophenyl)pyrrolidine-1-carboxylate

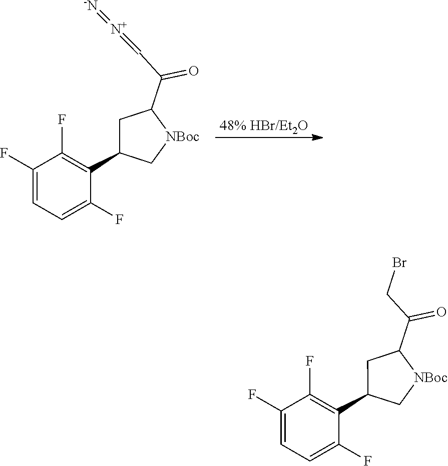

To a solution of (4R)-tert-butyl 2-(2-diazoacetyl)-4-(2,3,6-trifluorophenyl)pyrrolidine-1-carboxylate (6.91 g, 18.71 mmol) in diethyl ether (55 mL) was added 48% HBr (2.22 mL, 19.64 mmol) at 0-5° C. with stirring. After 5 min. the mixture was diluted with ethyl acetate (83 mL) and then washed with sodium bicarbonate solution. The organic phase was dried (MgSO₄), filtered, evaporated to dryness to give (4R)-tert-butyl 2-(2-bromoacetyl)-4-(2,3,6-trifluorophenyl)pyrrolidine-1-carboxylate as a light yellow oil. Yield: 6.4 g, 60%.

### Step 15: Diethyl 2-(2-((4R)-1-(tert-butoxycarbonyl)-4-(2,3,6-trifluorophenyl)pyrrolidin-2-yl)-2-oxoethyl)malonate

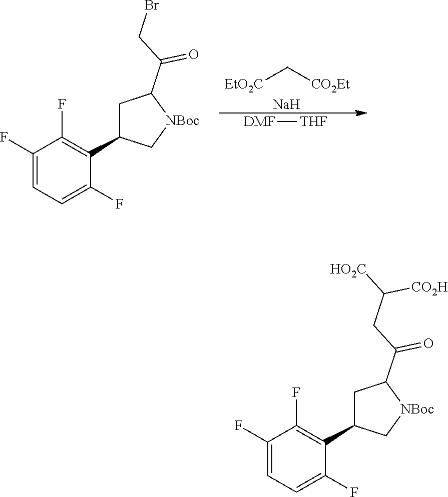

To a solution of diethyl malonate (3.47 mL, 22.74 mmol) in N,N-dimethyl formamide (28 mL) was added sodium hydride (60% in minar oil) (0.727 g, 18.9 mmol) with ice cooling and the solution was stirred for 30 min. Thereupon, (4R)-tert-butyl 2-(2-bromoacetyl)-4-(2,3,6-trifluorophenyl)pyrrolidine-1-carboxylate (6.4 g, 15.16 mmol) in dry tetrahydrofuran (14 mL) was added to the above reaction mixture with ice cooling and the mixture was stirred in the cold for 30 min. The reaction was then diluted with a mixture of ethyl acetate-petroleumether (2:1), washed with NaHSO₄ solution, dried over MgSO₄, filtered and evaporated to dryness. Chromatography in a mixture of ethyl acetate-petroleumether afforded the titled product as a light yellow oil. Yield: 5.44 g, 60%.

### Step 16: Diethyl 2-(2-oxo-2-((4R)-4-(2,3,6-trifluorophenyl)pyrrolidin-2-yl)ethyl)malonate hydrochloride

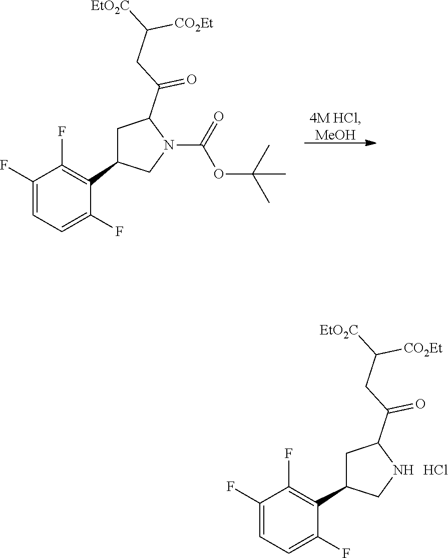

Diethyl 2-(2-((4R)-1-(tert-butoxycarbonyl)-4-(2,3,6-trifluorophenyl)pyrrolidin-2-yl)-2-oxoethyl)malonate (5.43 g, 10.83 mmol) was dissolved in 4 M HCl (40.6 mL, 162 mmol) in dioxane and the solution was stirred for 2 h. Thereupon, the mixture was diluted with diethyl ether (ca. 200 mL). The resulting crystals were collected, washed with diethyl ether and dried under vacuum to give diethyl 2-(2-oxo-2-((4R)-4-(2,3,6-trifluorophenyl)pyrrolidin-2-yl)ethyl)malonate hydrochloride as a white solid. Yield: 3.61 g, 64%.

### Step 17: diethyl (R)-2-((6-(2,3,6-trifluorophenyl)-3-thioxo-2,5,6,7-tetrahydro-3H-pyrrolo[1,2-c]imidazol-1-yl)methyl)malonate

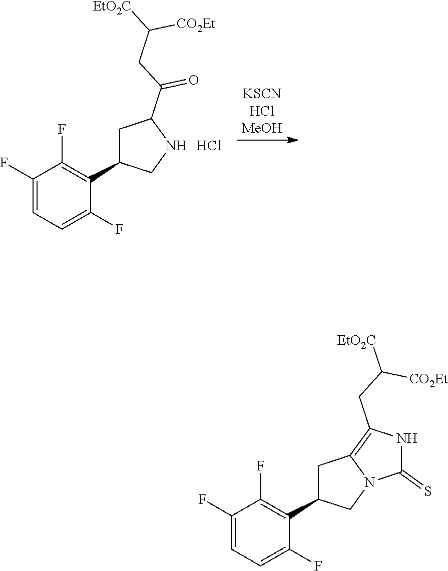 [SMILES: CCOC(=O)C(CC(=O)C1C[C@H](c2c(F)ccc(F)c2F)CN1)C(=O)OCC ; Cl ; CCOC(=O)C(Cc1[nH]c(=S)n2c1C[C@H](c1c(F)ccc(F)c1F)C2)C(=O)OCC]

A mixture of diethyl 2-(2-oxo-2-((4R)-4-(2,3,6-trifluorophenyl)pyrrolidin-2-yl)ethyl)malonate hydrochloride (3.6 g, 8.22 mmol), potassium isothiocyanate (1.039 g, 10.69 mmol) and cc. HCl (0.405 mL, 4.93 mmol) in abs. ethanol (86 mL) was stirred under reflux for 30 min. The suspension was then cooled to room temperature, evaporated to dryness and the residue was partitioned between dichloromethane and water. The organic phase was dried (MgSO₄), filtered and evaporated to dryness to give the titled product as a yellow foam. Yield: 3.31 g, 82% yield.

### Step 18: (R)-2-((6-(2,3,6-trifluorophenyl)-3-thioxo-2,5,6,7-tetrahydro-3H-pyrrolo[1,2-c]imidazol-1-yl)methyl)malonic acid

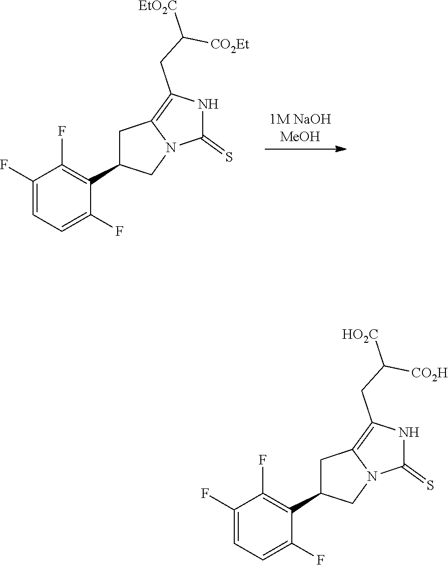 [SMILES: CCOC(=O)C(Cc1[nH]c(=S)n2c1C[C@H](c1c(F)ccc(F)c1F)C2)C(=O)OCC ; O=C(O)C(Cc1[nH]c(=S)n2c1C[C@H](c1c(F)ccc(F)c1F)C2)C(=O)O]

To a solution of (R)-diethyl 2-((6-(2,3,6-trifluorophenyl)-3-thioxo-3,5,6,7-tetrahydro-2H-pyrrolo[1,2-c]imidazol-1-yl)methyl)malonate (3.3 g, 7.46 mmol) in methanol (80 mL) was added 1 M sodium hydroxide solution (44.8 mL, 44.8 mmol) and the mixture was stirred at room temperature for 5 h. Thereupon, methanol was removed by vacuum, the residue was diluted with water and then acidified to pH=1 by addition of 2 M HCl solution with ice cooling. The resulting precipitate was collected by filtration washed with water and dried under vacuum to give (R)-2-((6-(2,3,-trifluorophenyl)-3-thioxo-3,5,6,7-tetrahydro-2H-pyrrolo[1,2-c]imidazol-1-yl)methyl)malonic acid as a yellow solid. Yield: 2.88 g, 85%.

### Step 19: (R)-3-(6-(2,3,6-trifluorophenyl)-3-thioxo-2,5,6,7-tetrahydro-3H-pyrrolo[1,2-c]imidazol-1-yl)propanoic acid

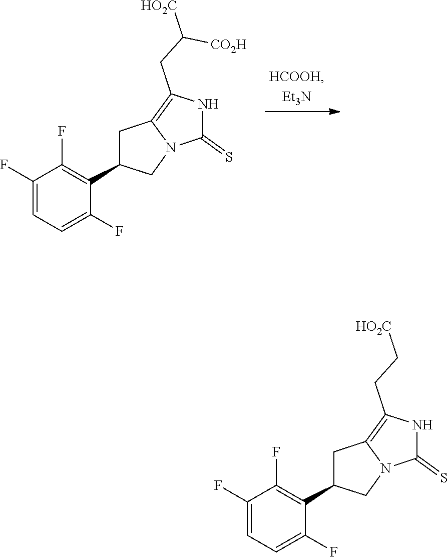 [SMILES: O=C(O)C(Cc1[nH]c(=S)n2c1C[C@H](c1c(F)ccc(F)c1F)C2)C(=O)O ; O=C(O)CCc1[nH]c(=S)n2c1C[C@H](c1c(F)ccc(F)c1F)C2]

To a solution of (R)-2-((6-(2,3,6-trifluorophenyl)-3-thioxo-3,5,6,7-tetrahydro-2H-pyrrolo[1,2-c]imidazol-1-yl)methyl)malonic acid (2.88 g, 7.45 mmol) in formic acid (8.58 mL, 224.0 mmol) was added triethylamine (12.47 mL, 89.0 mmol) dropwise with stirring (exothermic reaction), and then the resulting solution was stirred at 115° C. for 1 h. Thereupon, the mixture was treated with 1 M HCl (80 mL) and then aged for 30 min. The resultant solid was collected washed with water and dried under vacuum at 50° C. to give (R)-3-(6-(2,3,6-trifluorophenyl)-3-thioxo-3,5,6,7-tetrahydro-2H-pyrrolo[1,2-c]imidazol-1-yl)propanoic acid as a dark beige solid. Yield: 2.13 g, 75%.

### Intermediate 2: 3-((5aS,6aR)-5a-(5-chloro-2-fluorophenyl)-3-thioxo-2,3,5,5a,6,6a-hexahydrocyclopropa[3,4]pyrrolo[1,2-c]imidazol-1-yl)propanoic acid

### Step 1: ((1R,2S)-2-(aminomethyl)-2-(5-chloro-2-fluorophenyl)cyclopropyl)methanol

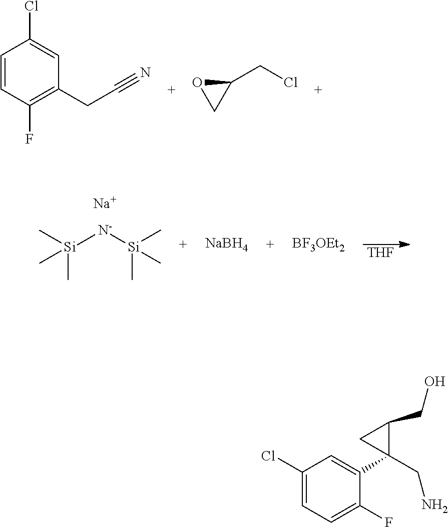

To a stirred solution of 2-(5-chloro-2-fluorophenyl)acetonitrile (10.0 g, 59.0 mmol) in dry tetrahydrofuran (100 mL), was added (R)-2-(chloromethyl)oxirane (5.53 mL, 70.8 mmol) at room temperature, under nitrogen. The reaction was then cooled to 15° C. and 2 M sodium bis(trimethylsilyl)amide in tetrahydrofuran (51.6 mL, 103.0 mmol) was added, dropwise at 15° C. over a period of 2 h. Thereupon, the thus obtained red mixture was allowed to warm up to room temperature and stirred for 2 h. The reaction was diluted with dry tetrahydrofuran (100 mL), cooled to 0° C., and then sodium borohydride (8.92 g, 236.0 mmol) was added followed by dropwise addition of boron trifluoride etherate (29.9 mL, 236.0 mmol). The mixture was allowed to warm up to room temperature and stirred overnight. The resulting pale yellow suspension was cooled to 0° C. and carefully quenched with 2M HCl (177 mL, 354 mmol). The tetrahydrofuran was then evaporated off and the aqueous phase was washed with diethyl ether. The pH of the aqueous phase was set to pH=10 by adding 3 M sodium hydroxide and then extracted with dichloromethane. The organic phase was dried over MgSO₄, filtered and evaporated to dryness under vacuum to leave a yellow oil. (Yield: 12.95 g, 81%).

### Step 2: tert-butyl (((1S,2R)-1-(5-chloro-2-fluorophenyl)-2-(hydroxymethyl)cyclopropyl)methyl)carbamate

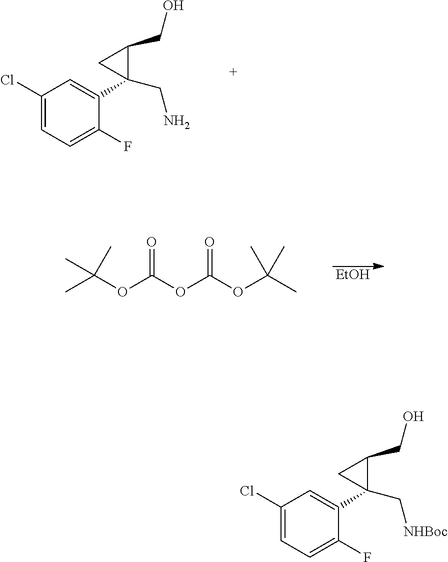 [SMILES: "EtOH" ; NC[C@@]1(c2cc(Cl)ccc2F)C[C@H]1CO ; CC(C)(C)OC(=O)OC(=O)OC(C)(C)C ; CC(C)(C)OC(=O)NC[C@@]1(c2cc(Cl)ccc2F)C[C@H]1CO]

To an ice-cold solution of ((1R,2S)-2-(aminomethyl)-2-(5-chloro-2-fluorophenyl)cyclopropyl)methanol (12.95 g, 56.4 mmol) in ethanol (205 mL) was added di-tert-butyl dicarbonate (12.31 g, 56.4 mmol). The solution was stirred at room temperature for 3 h and then the solvent was evaporated off under vacuum. The resulting yellow oil was purified by chromatography (petroleum ether-ethyl acetate). The product was isolated as a colorless foam. (Yield: 13.65 g, 62%).

### Step 3: tert-butyl (1S,5R)-1-(5-chloro-2-fluorophenyl)-4-hydroxy-3-azabicyclo[3.1.0]hexane-3-carboxylate

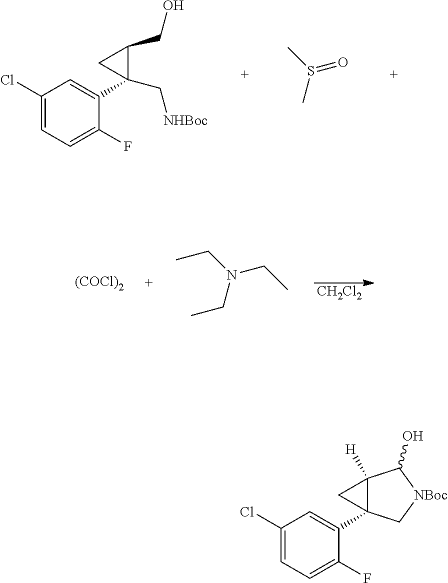

To a stirred solution of oxalyl dichloride (3.99 mL, 45.50 mmol) in dry dichloromethane (94 mL), was added dropwise a solution of dimethylsulfoxide (6.46 mL, 91.0 mmol) in dry dichloromethane (19 mL) at −78° C. The reaction mixture was stirred in the cold for 15 min, and then a solution of tert-butyl tert-butyl (((1S,2R)-1-(5-chloro-2-fluorophenyl)-2-(hydroxymethyl)cyclopropyl)methyl)carbamate (13.65 g, 41.4 mmol) in dry dichloromethane (38 mL) was added dropwise. The mixture was stirred at −78° C. for 1 h and then triethylamine (28.8 mL, 207.0 mmol) was added. The reaction was allowed to warm up gradually to room temperature and stirred at room temperature for 2 h. Thereupon, the mixture was washed three times with water, dried over MgSO₄, filtered and evaporated to dryness to give a yellow oil. (Yield: 14.0 g, 83%).

### Step 4: tert-butyl (1S,5R)-1-(5-chloro-2-fluorophenyl)-4-cyano-3-azabicyclo[3.1.0]hexane-3-carboxylate

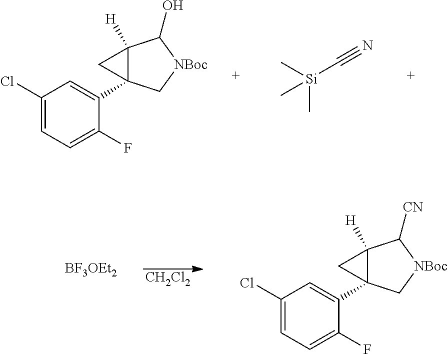

To a stirred solution of tert-butyl (1S,5R)-1-(5-chloro-2-fluorophenyl)-4-hydroxy-3-azabicyclo[3.1.0]hexane-3-carboxylate (13.6 g, 41.5 mmol) in dry dichloromethane (210 mL) was added trimethylsilanecarbonitrile (14.85 ml, 111.0 mmol) at room temperature under nitrogen. The solution was then cooled to −78° C. and boron trifluoride etherate (15.42 mL, 111.0 mmol) was added dropwise. The reaction mixture was stirred in the cold for 4 h., and then saturated solution of sodium bicarbonate was added and allowed to warm up to room temperature. The organic phase was separated and aqueous phase was extracted with dichloromethane. The combined organic phases were dried over MgSO₄, filtered and evaporated to dryness to leave a yellow oil. (Yield: 13.1 g, 80%).

### Step 5: (1R,5S)-3-(tert-butoxycarbonyl)-5-(5-chloro-2-fluorophenyl)-3-azabicyclo[3.1.0]hexane-2-carboxylic acid

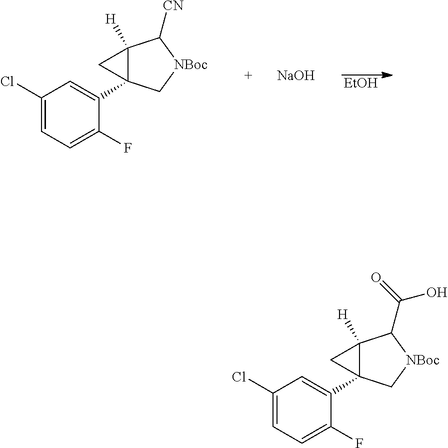 [SMILES: "EtOH" ; CC(C)(C)OC(=O)N1C[C@@]2(c3cc(Cl)ccc3F)C[C@H]2C1C#N ; "NaOH" ; CC(C)(C)OC(=O)N1C[C@@]2(c3cc(Cl)ccc3F)C[C@H]2C1C(=O)O]

To a stirred solution of tert-butyl (1S,5R)-4-cyano-1-(5-chloro-2-fluorophenyl)-3-azabicyclo[3.1.0]hexane-3-carboxylate (13.1 g, 38.9 mmol) in ethanol (135 mL), was added a solution of 3 M sodium hydroxide (64.8 mL, 194.0 mmol) at room temperature. The solution was heated at 80° C. for 5 h and then was cooled to room temperature. Thereupon, ethanol was evaporated off and the aqueous phase was acidified with 2 M HCl solution and then extracted with a mixture of dichloromethane-isopropanol (7:3). The organic phase was dried over MgSO₄, filtered and evaporated to dryness to leave a yellow solid. (Yield: 11.56 g, 71%).

### Step 6: (1S,5R)-tert-butyl 1-(5-chloro-2-fluorophenyl)-4-(2-diazoacetyl)-3-azabicyclo[3.1.0]hexane-3-carboxylate

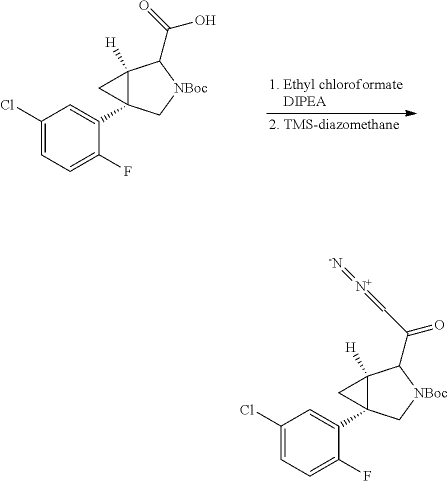

To a solution of (1R,5S)-3-(tert-butoxycarbonyl)-5-(5-chloro-2-fluorophenyl)-3-azabicyclo[3.1.0]hexane-2-carboxylic acid (11 g, 30.9 mmol) and N-ethyl-N-isopropylpropan-2-amine (DIPEA) (9.45 mL, 54.1 mmol) in dry tetrahydrofuran (114 mL) was added ethyl chloroformate (4.45 mL, 46.40 mmol) at 0-5° C. The mixture was stirred for 4 h in the cold, and then diluted with acetonitrile (57 mL) followed by addition of 2 M (diazomethyl)trimethylsilane (30.9 mL 61.80 mmol) in diethyl ether. The stirring was continued for additional 3 h at 0-5° C. and the mixture was allowed to warm up naturally overnight with stirring under N₂. Thereupon, the solvents were removed under vacuum and the residue was purified by column chromatography in a mixture of petroleumether-ethyl acetate to give (1S,5R)-tert-butyl 1-(5-chloro-2-fluorophenyl)-4-(2-diazoacetyl)-3-azabicyclo[3.1.0]hexane-3-carboxylate as a light yellow oil. Yield: 8.38 g, 60%.

### Step 7: tert-butyl (1S,5R)-4-(2-bromoacetyl)-1-(5-chloro-2-fluorophenyl)-3-azabicyclo[3.1.0]hexane-3-carboxylate

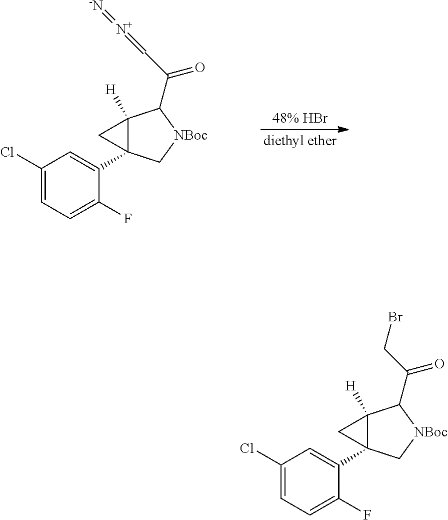

To a solution of 1-(5-chloro-2-fluorophenyl)-4-(2-diazoacetyl)-3-azabicyclo[3.1.0]hexane-3-carboxylate (8.30 g, 21.85 mmol) in diethyl ether (64 mL) was added 48% HBr (2.60 mL, 22.95 mmol) at 0-5° C. with stirring. After 5 min. the mixture was diluted with ethyl acetate (97 mL) and then washed with sodium bicarbonate solution. The organic phase was dried (MgSO₄), filtered, evaporated to dryness to give tert-butyl (1S,5R)-4-(2-bromoacetyl)-1-(5-chloro-2-fluorophenyl)-3-azabicyclo[3.1.0]hexane-3-carboxylate as a light yellow oil. Yield: 9.25 g, 83%.

### Step 8: diethyl 2-(2-((1R,5S)-3-(tert-butoxycarbonyl)-5-(5-chloro-2-fluorophenyl)-3-azabicyclo[3.1.0]hexan-2-yl)-2-oxoethyl)malonate

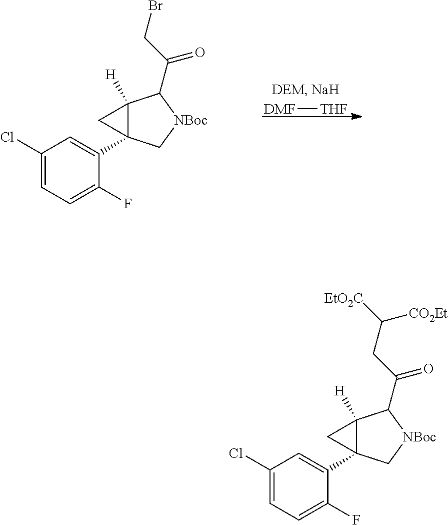

To a solution of diethyl malonate (4.89 mL, 32.10 mmol) in N,N-dimethyl formamide (40 mL) was added sodium hydride (60% in minar oil) (1.026 g, 25.70 mmol) with ice cooling and the solution was stirred for 30 min. Thereupon, tert-butyl (1S,5R)-4-(2-bromoacetyl)-1-(5-chloro-2-fluorophenyl)-3-azabicyclo[3.1.0]hexane-3-carboxylate (9.25 g, 21.38 mmol) in dry tetrahydrofuran (20.0 mL) was added to the above reaction mixture with ice cooling and the mixture was stirred in the cold for 30 min. The reaction was then diluted with a mixture of ethyl acetate-petroleumether (2:1), washed with NaHSO₄ solution, dried over MgSO₄, filtered and evaporated to dryness. Chromatography in a mixture of ethyl acetate-petroleumether afforded the titled product as a yellow oil. Yield: 9.37 g, 72%.

### Step 9: diethyl 2-(2-((1R,5S)-5-(5-chloro-2-fluorophenyl)-3-azabicyclo[3.1.0]hexan-2-yl)-2-oxoethyl)malonate hydrochloride

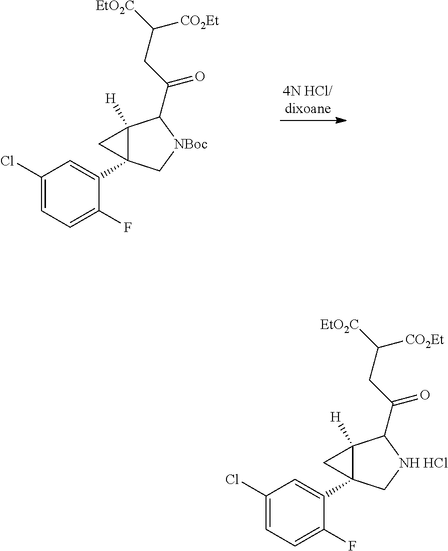

Diethyl 2-(2-((1R,5S)-3-(tert-butoxycarbonyl)-5-(5-chloro-2-fluorophenyl)-3-azabicyclo[3.1.0]hexan-2-yl)-2-oxoethyl)malonate (9.3 g, 18.17 mmol) was dissolved in 4 M HCl (68.1 mL, 272 mmol) in dioxane and the solution was stirred for 2 h. Thereupon, the mixture was diluted with diethyl ether (ca. 180 mL) The resulting crystals were collected, washed with diethyl ether and dried under vacuum at 50° C. to give diethyl 2-(2-((1R,5S)-5-(5-chloro-2-fluorophenyl)-3-azabicyclo[3.1.0]hexan-2-yl)-2-oxoethyl)malonate hydrochloride as a yellow solid. Yield: 8.3 g, 98%.

### Step 10: diethyl 2-(((5aS,6aR)-5a-(5-chloro-2-fluorophenyl)-3-thioxo-2,3,5,5a,6,6a-hexahydrocyclopropa[3,4]pyrrolo[1,2-c]imidazol-1-yl)methyl)malonate

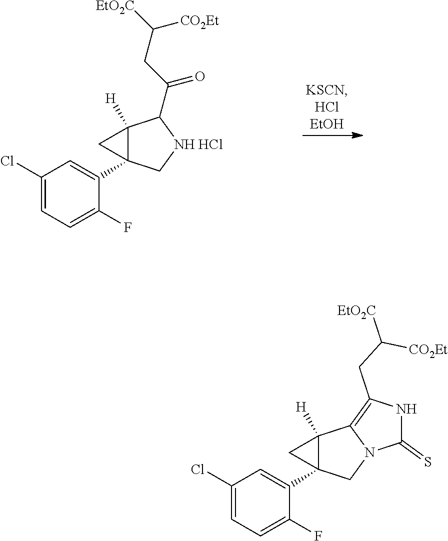

A mixture of diethyl diethyl 2-(2-((1R,5S)-5-(5-chloro-2-fluorophenyl)-3-azabicyclo[3.1.0]hexan-2-yl)-2-oxoethyl)malonate hydrochloride (8.11 g, 18.07 mmol), potassium isothiocyanate (2.283 g, 23.49 mmol) and cc. HCl (0.89 mL, 10.84 mmol) in abs. ethanol (187 mL) was stirred under reflux for 30 min. The suspension was then cooled to room temperature, diluted with water, stirred for 30 min., and then ethanol was evaporated off. The aqueous phase was extracted with dichloromethane. The organic phase was dried (MgSO₄), filtered and evaporated to dryness to give the titled product as a yellow foam. Yield: 8.2 g, 75%.

### Step 11: 2-(((5aS,6aR)-5a-(5-chloro-2-fluorophenyl)-3-thioxo-2,3,5,5a,6,6a-hexahydrocyclopropa[3,4]pyrrolo[1,2-c]imidazol-1-yl)methyl)malonic acid

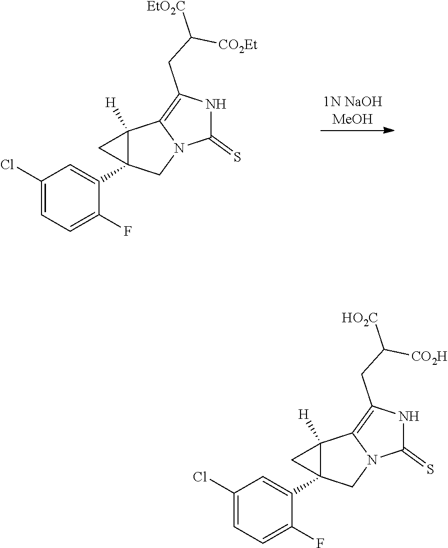 [SMILES: CCOC(=O)C(Cc1[nH]c(=S)n2c1[C@@H]1C[C@]1(c1cc(Cl)ccc1F)C2)C(=O)OCC ; O=C(O)C(Cc1[nH]c(=S)n2c1[C@@H]1C[C@]1(c1cc(Cl)ccc1F)C2)C(=O)O]

To a solution of diethyl 2-(((5aS,6aR)-5a-(5-chloro-2-fluorophenyl)-3-thioxo-2,3,5,5a,6,6a-hexahydrocyclopropa[3,4]pyrrolo[1,2-c]imidazol-1-yl)methyl)malonate (8.1 g, 17.88 mmol) in methanol (190 mL) was added 1 M sodium hydroxide solution (107 mL, 107 mmol) and the mixture was stirred at room temperature overnight. Thereupon, methanol was removed by vacuum, the residue was diluted with water then acidified to pH=1 by addition of 2 M HCl solution with ice cooling. The mixture was then extracted with a mixture of dichloromethane-isopropanol (7:3), the organic phase was dried over MgSO₄, filtered to give 2-(((5aS,6aR)-5a-(5-chloro-2-fluorophenyl)-3-thioxo-2,3,5,5a,6,6a-hexahydrocyclopropa[3,4]pyrrolo[1,2-c]imidazol-1-yl)methyl)malonic acid as a yellow foam. Yield: 6.2 g, 87%.

### Step 12: 3-((5aS,6aR)-5a-(5-chloro-2-fluorophenyl)-3-thioxo-2,3,5,5a,6,6a-hexahydrocyclopropa[3,4]pyrrolo[1,2-c]imidazol-1-yl)propanoic acid

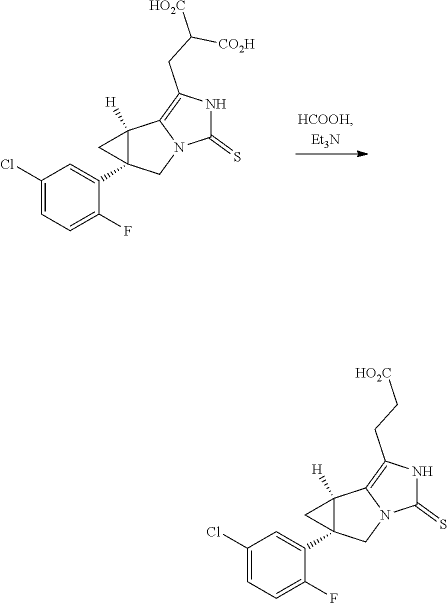 [SMILES: O=C(O)C(Cc1[nH]c(=S)n2c1[C@@H]1C[C@]1(c1cc(Cl)ccc1F)C2)C(=O)O ; O=C(O)CCc1[nH]c(=S)n2c1[C@@H]1C[C@]1(c1cc(Cl)ccc1F)C2]

To a solution of 2-(((5aS,6aR)-5a-(5-chloro-2-fluorophenyl)-3-thioxo-2,3,5,5a,6,6a-hexahydrocyclopropa[3,4]pyrrolo[1,2-c]imidazol-1-yl)methyl)malonic acid (6.2 g, 15.62 mmol) in formic acid (17.98 mL, 469 mmol) was added triethylamine (26.1 mL, 187 mmol) dropwise with stirring (exothermic reaction), and then the resulting solution was stirred at 115° C. for 1 h. Thereupon, the mixture was cooled to 0° C. and 1 N HCl (234 mL, 234 mmol) was added, and then aged for 90 min. The obtained solid mass was separated by decantation, and then dissolved in a mixture of dichloromethane-isopropanol (7:3), dried over MgSO₄, filtered and evaporated to dryness. The crude product was purified by column chromatography (dichloromethane-methanol) followed by crystallization from isopropyl acetate to give 3-((5aS,6aR)-5a-(5-chloro-2-fluorophenyl)-3-thioxo-2,3,5,5a,6,6a-hexahydrocyclopropa[3,4]pyrrolo[1,2-c]imidazol-1-yl)propanoic acid as a white solid. Yield: 2.62 g, 45%.

### Intermediate 3: 3-((5aS,6aR)-5a-(3-chloro-2,6-difluorophenyl)-3-thioxo-2,3,5,5a,6,6a-hexahydrocyclopropa[3,4]pyrrolo[1,2-c]imidazol-1-yl)propanoic acid

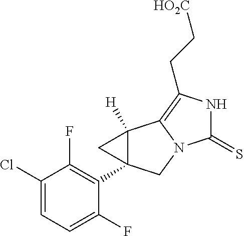 [SMILES: O=C(O)CCc1[nH]c(=S)n2c1[C@@H]1C[C@]1(c1c(F)ccc(Cl)c1F)C2]

Compound was prepared analogous manner to Intermediate 2 from 2-(3-chloro-2,6-difluorophenyl)acetonitrile and isolated as an off-white powder.

### Method A:

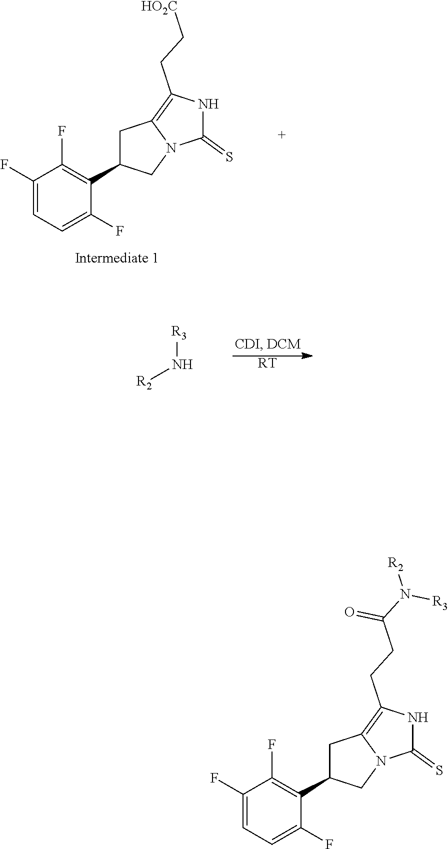

To a stirred suspension of intermediate 1 (400 mg, 1.168 mmol) in anhydrous dichloromethane (12 mL) was added 1,1′-carbonyldiimidazole (227 mg, 1.402 mmol) portionwise. The reaction mixture was stirred at room temperature for 30 min. The obtained solution was treated with R₂R₃NH (2.337 mmol) and stirred for 2 h at room temperature. Thereupon, the mixture was washed with sodium bicarbonate solution, the organic phase was dried over MgSO₄, filtered, and then evaporated to dryness. The product was purified by chromatography.

### Method B:

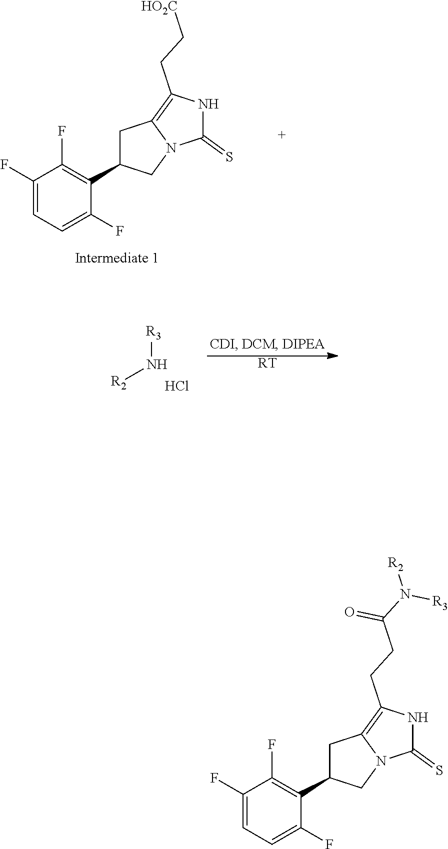

To a stirred suspension of intermediate 1 (400 mg, 1.168 mmol) in anhydrous dichloromethane (12 mL) was added 1,1′-carbonyldiimidazole (227 mg, 1.402 mmol) portionwise. The reaction mixture was stirred at room temperature for 30 min. The obtained solution was treated with R₂R₃NH.HCl (2.337 mmol) followed by addition of N-ethyldiisopropylamine (0.408 mL, 2.337 mmol) and stirred for 2 h at room temperature. Thereupon, the mixture was washed with sodium bicarbonate solution, the organic phase was dried over MgSO₄, filtered, and then evaporated to dryness. The product was purified by chromatography.

### Method C:

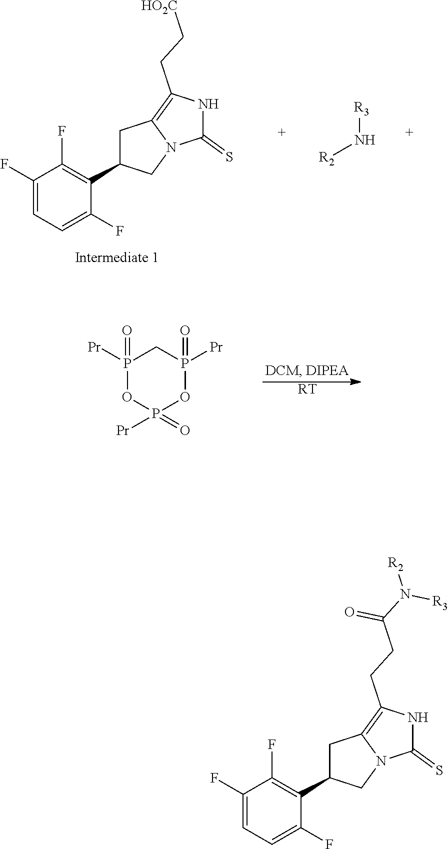

To a stirred suspension of intermediate 1 (400 mg, 1.168 mmol) in anhydrous dichloromethane (12 mL) was added R₂R₃NH.HCl (2.337 mmol) followed by addition of N-ethyldiisopropylamine (0.245 mL, 1.402 mmol) and 1-propanephosphonicacidcyclicanhydride (0.871 mL, 1.402 mmol). The reaction mixture was stirred for 2 h at room temperature. Thereupon, the mixture was washed with sodium bicarbonate solution, the organic phase was dried over MgSO₄, filtered, and then evaporated to dryness. The product was purified by chromatography.

### General Protocol for Reduction of Amides to Amines:

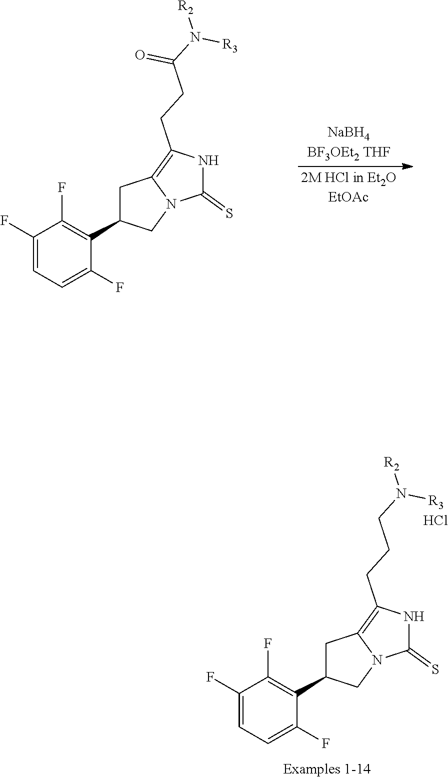

To a stirred suspension of the starting amide (1.00 mmol) in anhydrous tetrahydrofuran (4.2 mL) was added sodium borohydride (189 mg, 5.01 mmol) and the reaction was then cooled to 0° C. Thereupon, a solution of boron trifluoride etherate (0.635 mL, 5.01) in anhydrous tetrahydrofuran (1.9 mL) was added dropwise and the reaction was allowed to stir at room temperature for 2 h. Thereupon, the reaction was cooled again to 0° C. and quenched with 1 M HCl (˜1.4 mL), followed by addition of 2 M HCl (˜1.2 mL, to pH=1). The mixture was then allowed to warm up to room temperature and heated at reflux for 30 min. Thereupon, the mixture was cooled to room temperature, diluted with water, and then tetrahydrofuran was evaporated off. The aqueous phase was basified by addition of 1M NaOH solution and then extracted with dichloromethane. The organic phase was dried over MgSO₄, filtered and evaporated to dryness to give the desired amines.

The amines were converted to HCl salt in ethyl acetate on the reaction with 10 equivalents of 2M HCl in diethyl ether.

### Example 1: (R)-1-(3-morpholinopropyl)-6-(2,3,6-trifluorophenyl)-2,5,6,7-tetrahydro-3H-pyrrolo[1,2-c]imidazole-3-thione hydrochloride

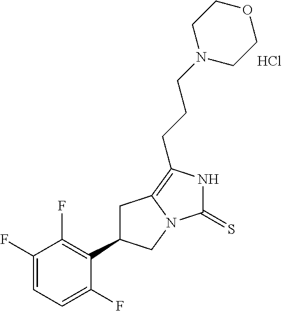 [SMILES: Fc1ccc(F)c([C@H]2Cc3c(CCCN4CCOCC4)[nH]c(=S)n3C2)c1F ; Cl]

Compound was prepared from Intermediate 1 and morpholine by Method A and isolated as a light beige solid.

¹H NMR (DMSO_{d6}): 11.85 (1H, br s), 11.10 (1H, br s), 7.48 (1H, m), 7.19 (1H, m), 4.45 (1H, quin, J=8.7 Hz), 4.15 (1H, dd, J=11.4, 9.2 Hz), 3.93 (2H, br dd, J=12.4, 2.7 Hz), 3.80 (2H, br t, J=12.2 Hz), 3.74 (1H, dd, J=11.6, 8.2 Hz), 3.38 (2H, m), 3.32 (1H, d, J=8.9, 15.5 Hz), 3.03 (4H, m), 2.94 (1H, dd, J=15.6, 8.4 Hz), 2.45 (2H, br t, J=7.5 Hz), 1.97 (2H, m).

¹³C NMR (DMSO_{d6}): 156.9, 156.9, 155.3, 155.3, 149.1, 149.1, 149, 149, 147.6, 147.6, 147.5, 147.5, 147.4, 147.4, 147.3, 145.9, 145.9, 145.9, 145.8, 128, 118.8, 118.7, 118.7, 118.6, 118.1, 116.6, 116.5, 116.4, 116.4, 112, 112, 112, 112, 111.9, 111.8, 111.8, 111.8, 63.1, 55.1, 50.9, 48.3, 35.7, 29, 21.5, 21.3.

### Example 2: (R)-1-(3-aminopropyl)-6-(2,3,6-trifluorophenyl)-2,5,6,7-tetrahydro-3H-pyrrolo[1,2-c]imidazole-3-thione

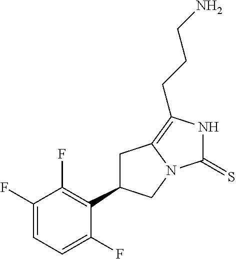 [SMILES: NCCCc1[nH]c(=S)n2c1C[C@H](c1c(F)ccc(F)c1F)C2]

Compound was prepared from Intermediate 1 and ammonia by Method A and isolated as a white solid.

¹H NMR (DMSO_{d6}): 7.47 (1H, m), 7.18 (1H, m), 4.45 (1H, quin, J=8.6 Hz), 4.14 (1H, dd, J=11.4, 9.1 Hz), 3.72 (1H, dd, J=11.6, 7.9 Hz), 3.27 (1H, dd, J=15.6, 9.2 Hz), 2.90 (1H, dd, J=8.2, 15.7 Hz), 2.56 (2H, t, J=7.0 Hz), 2.39 (2H, t, J=7.4 Hz), 1.62 (2H, quin, J=7.2 Hz).

¹³C NMR (DMSO_{d6}): 156.9, 156.9, 156.9, 156.9, 155.3, 155.3, 155.3, 155.3, 155, 149.1, 149, 149, 148.9, 147.5, 147.5, 147.4, 147.4, 147.3, 147.3, 146, 145.9, 145.9, 145.8, 127.5, 119.5, 119.1, 119, 119, 118.9, 116.5, 116.4, 116.4, 116.3, 112, 111.8, 48.4, 40.0, 35.6, 30.3, 29.1, 21.3.

### Example 3: (R)-1-(3-(((tetrahydro-2H-pyran-4-yl)methyl)amino)propyl)-6-(2,3,6-trifluorophenyl)-2,5,6,7-tetrahydro-3H-pyrrolo[1,2-c]imidazole-3-thione

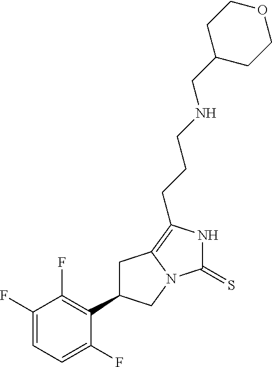 [SMILES: Fc1ccc(F)c([C@H]2Cc3c(CCCNCC4CCOCC4)[nH]c(=S)n3C2)c1F]

Compound was prepared from Intermediate 1 and (tetrahydro-2H-pyran-4-yl)methanamine by Method A and isolated as an off-white solid.

¹H NMR (DMSO_{d6}): 11.32 (1H, br), 7.47 (1H, m), 7.18 (1H, m), 4.44 (1H, quin, J=8.5 Hz), 4.14 (1H, dd, J=11.5, 9.2 Hz), 3.80 (2H, br dd, J=11.1, 4.2 Hz), 3.73 (1H, dd, J=11.7, 7.8 Hz), 3.27 (1H, dd, J=9.5, 15.5 Hz), 3.24 (2H, m), 2.87 (1H, dd, J=15.6, 8.1 Hz), 2.5 (2H, m), 2.39 (4H, m), 1.66 (2H, quin, J=7.2 Hz), 1.58 (3H, m), 1.12 (2H, m).

¹³C NMR (DMSO_{d6}): 156.9, 156.9, 156.9, 156.9, 155.3, 155.3, 155.3, 155.3, 155, 149.1, 149, 149, 148.9, 147.5, 147.5, 147.4, 147.4, 147.3, 147.3, 146, 145.9, 145.9, 145.8, 127.6, 119.5, 119.1, 119, 119, 118.9, 116.5, 116.4, 116.4, 116.3, 112, 111.8, 66.9, 55.2, 48.4, 48.3, 35.7, 34.6, 31, 29.1, 27.5, 21.7.

### Example 4: (R)-1-(3-aminopropyl)-6-(2,3,6-trifluorophenyl)-2,5,6,7-tetrahydro-3H-pyrrolo[1,2-c]imidazole-3-thione hydrochloride

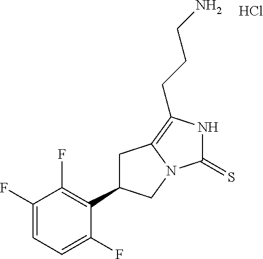 [SMILES: NCCCc1[nH]c(=S)n2c1C[C@H](c1c(F)ccc(F)c1F)C2 ; Cl]

Compound was prepared from Intermediate 1 and ammonia by Method A and isolated as a yellow solid.

¹H NMR (DMSO_{d6}): 11.85 (1H, s), 7.95 (2H, br s), 7.48 (1H, m), 7.19 (1H, m), 4.46 (1H, quin, J=8.6 Hz), 4.15 (1H, dd, J=11.4, 9.2 Hz), 3.73 (1H, dd, J=11.6, 7.9 Hz), 3.30 (1H, dd, J=15.6, 9.2 Hz), 2.90 (1H, dd, J=15.6, 8.2 Hz), 2.73 (2H, m), 2.45 (2H, t. J=7.1 Hz), 1.81 (2H, quin, J=7.5 Hz).

¹³C NMR (DMSO_{d6}): 156.9, 156.9, 155.3, 155.3, 149.1, 149, 149, 148.9, 147.5, 147.5, 147.5, 147.4, 147.4, 147.3, 147.3, 146, 145.9, 145.9, 145.8, 127.9, 118.9, 118.9, 118.8, 118.7, 118.4, 116.5, 116.5, 116.4, 116.3, 112, 112, 112, 112, 111.9, 111.8, 111.8, 111.8, 48.4, 38, 35.6, 29.1, 25.8, 21.

### Example 5: (R)-1-(3-((tetrahydro-2H-pyran-4-yl)amino)propyl)-6-(2,3,6-trifluorophenyl)-2,5,6,7-tetrahydro-3H-pyrrolo[1,2-c]imidazole-3-thione

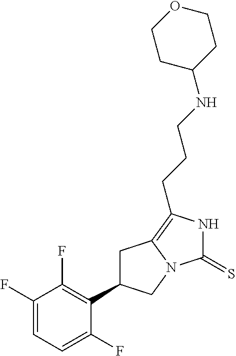 [SMILES: Fc1ccc(F)c([C@H]2Cc3c(CCCNC4CCOCC4)[nH]c(=S)n3C2)c1F]

Compound was prepared from Intermediate 1 and tetrahydro-2H-pyran-4-amine by Method A and isolated as a white solid.

¹H NMR (DMSO_{d6}): 11.80 (1H, br s), 7.47 (1H, m), 7.18 (1H, t, J=9.6 Hz), 4.45 (1H, quin, J=8.5 Hz), 4.14 (1H, dd, J=11.5, 9.2 Hz), 3.80 (2H, ddt, J=11.1, 7.4, 3.5, 3.5 Hz), 3.73 (1H, dd, J=11.6, 7.8 Hz), 3.28 (1H, dd, J=15.6, 9.4 Hz), 3.25 (2H, m), 2.87 (1H, dd, J=15.6, 7.9 Hz), 2.59 (1H, m), 2.53 (2H, m), 2.40 (2H, br t, J=7.4 Hz), 1.72 (2H, m), 1.65 (2H, quin, J=7.2 Hz), 1.21 (2H, m).

¹³C NMR (DMSO_{d6}): 156.9, 156.9, 156.9, 156.9, 155.3, 155.3, 155.3, 155.3, 155, 149.1, 149, 149, 148.9, 147.5, 147.5, 147.4, 147.4, 147.3, 147.3, 146, 145.9, 145.9, 145.8, 127.6, 119.5, 119.1, 119, 119, 118.9, 116.5, 116.4, 116.4, 116.3, 112, 111.8, 65.8, 53.4, 48.4, 44.6, 35.6, 32.8, 29.1, 27.9, 21.8.

### Example 6: (R)-1-(3-morpholinopropyl)-6-(2,3,6-trifluorophenyl)-2,5,6,7-tetrahydro-3H-pyrrolo[1,2-c]imidazole-3-thione

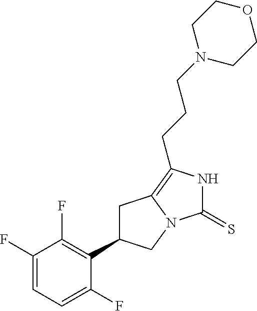 [SMILES: Fc1ccc(F)c([C@H]2Cc3c(CCCN4CCOCC4)[nH]c(=S)n3C2)c1F]

Compound was prepared from Intermediate 1 and morpholine by Method A and isolated as a beige solid.

¹H NMR (DMSO_{d6}): 11.78 (1H, s), 7.47 (1H, m), 7.18 (1H, m), 4.44 (1H, quin, J=8.5 Hz), 4.14 (1H, dd, J=11.4, 9.2 Hz), 3.73 (1H, dd, J=11.6, 7.9 Hz), 3.54 (4H, m), 3.28 (1H, br dd, J=15.6, 9.2 Hz), 2.88 (1H, br dd, J=15.4, 8.1 Hz), 2.36 (2H, br t, J=7.4 Hz), 2.30 (4H, br s), 2.23 (2H, br t, J=7.0 Hz), 1.67 (2H, quin, J=7.3 Hz).

¹³C NMR (DMSO_{d6}): 156.9, 156.9, 156.9, 156.9, 155.3, 155.3, 155.3, 155.3, 155, 149.1, 149, 149, 148.9, 147.5, 147.5, 147.4, 147.4, 147.3, 147.3, 146, 145.9, 145.9, 145.8, 127.6, 119.4, 119.1, 119, 119, 118.9, 116.5, 116.4, 116.4, 116.3, 112, 111.8, 66.1, 57, 53.1, 48.3, 35.7, 29.0, 24.3, 21.7.

### Example 7: (R)-1-(3-((R)-3-fluoropyrrolidin-1-yl)propyl)-6-(2,3,6-trifluorophenyl)-2,5,6,7-tetrahydro-3H-pyrrolo[1,2-c]imidazole-3-thione hydrochloride

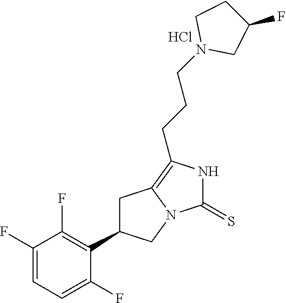 [SMILES: Fc1ccc(F)c([C@H]2Cc3c(CCCN4CC[C@@H](F)C4)[nH]c(=S)n3C2)c1F ; Cl]

Compound was prepared from Intermediate 1 and (R)-3-fluoropyrrolidine hydrochloride by Method B and isolated as a pale brown powder.

¹H NMR (DMSO_{d6}): 11.96, 11.45, 10.98 (2H, m), 7.49 (1H, m), 7.19 (1H, m), 5.44 (1H, d, J=53 Hz), 4.48 (1H, quin, J=8.6 Hz), 4.19 (1H, br t, J=10.2 Hz), 3.90-3.73 (2H, m), 3.67 (1H, m), 3.44-3.30 (2H, m), 3.22-3.05 (3H, m), 2.95 (1H, dd, J=8.2, 15.7 Hz), 2.48 (2H, m), 2.35-2.0 (2H, m), 1.96 (2H, m).

¹³C NMR (DMSO_{d6}): 156.9, 156.9, 155.3, 155.3, 149.1, 149, 149, 149, 147.7, 147.6, 147.5, 147.4, 147.4, 147.3, 146, 145.9, 145.9, 145.8, 118.7, 118.6, 118.6, 118.5, 116.6, 116.5, 116.5, 116.4, 112, 112, 112, 112, 111.9, 111.9, 111.8, 111.8, 92.6, 92.5, 92.4, 91.4, 91.3, 91.2, 58.6, 54.4, 53.8, 51.5, 48.5, 35.8, 31.6, 30.6, 29, 23.8, 21.4.

### Example 8: (R)-1-(3-(dimethylamino)propyl)-6-(2,3,6-trifluorophenyl)-2,5,6,7-tetrahydro-3H-pyrrolo[1,2-c]imidazole-3-thione

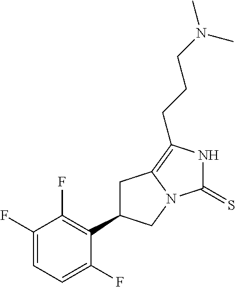 [SMILES: CN(C)CCCc1[nH]c(=S)n2c1C[C@H](c1c(F)ccc(F)c1F)C2]

Compound was prepared from Intermediate 1 and dimethylamine hydrochloride by Method B and isolated as an off-white solid.

¹H NMR (DMSO_{d6}): 11.78 (1H, br s), 7.48 (1H, m), 7.18 (1H, m), 4.45 (1H, quin, J=8.6 Hz), 4.14 (1H, dd, J=11.6, 9.1 Hz), 3.73 (1H, dd, J=11.7, 7.9 Hz), 3.30 (1H, dd, J=9.3, 15.7 Hz), 2.89 (1H, dd, J=15.6, 8.2 Hz), 2.36 (2H, t, J=7.6 Hz), 2.30 (2H, br m), 2.21 (6H, br s), 1.68 (2H, m).

¹³C NMR (DMSO_{d6}): 156.9, 156.9, 156.9, 156.9, 155.3, 155.3, 155.3, 155.3, 155, 149.1, 149, 149, 148.9, 147.5, 147.5, 147.4, 147.4, 147.3, 147.3, 146, 145.9, 145.9, 145.8, 127.8, 119.3, 119.1, 119, 119, 118.9, 116.5, 116.4, 116.4, 116.3, 112, 111.8, 57.7, 48.4, 44.6, 35.7, 29.0, 25.1, 21.6.

### Example 9: (R)-1-(3-(((S)-tetrahydro-2H-pyran-3-yl)amino)propyl)-6-(2,3,6-trifluorophenyl)-2,5,6,7-tetrahydro-3H-pyrrolo[1,2-c]imidazole-3-thione

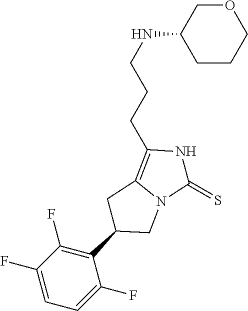 [SMILES: Fc1ccc(F)c([C@H]2Cc3c(CCCN[C@H]4CCCOC4)[nH]c(=S)n3C2)c1F]

Compound was prepared from Intermediate 1 and (S)-tetrahydro-2H-pyran-3-amine hydrochloride by Method B and isolated as an off-white solid.

¹H NMR (DMSO_{d6}): 11.79 (1H, m), 7.47 (1H, m), 7.17 (1H, m), 4.44 (1H, quin, J=8.5 Hz), 4.14 (1H, dd, J=11.6, 9.1 Hz), 3.75 (1H, m), 3.72 (1H, dd, J=11.6, 7.9 Hz), 3.66 (1H, dt, J=11.1, 3.9 Hz), 3.27 (1H, br dd, J=15.5, 9.3 Hz), 3.22 (1H, td, J=10.8, 2.6 Hz), 2.96 (1H, br t, J=9.9 Hz), 2.88 (1H, dd, J=15.6, 8.1 Hz), 2.53 (1H, m), 2.47 (2H, m), 2.38 (2H, t, J=7.3 Hz), 1.88 (1H, m), 1.67-1.53 (3H, m), 1.44 (1H, m), 1.21 (1H, m).

¹³C NMR (DMSO_{d6}): 156.9, 156.9, 156.9, 156.9, 155.3, 155.3, 155.3, 155.3, 155, 149.1, 149, 149, 148.9, 147.5, 147.5, 147.4, 147.4, 147.3, 147.3, 146, 145.9, 145.9, 145.8, 127.6, 119.5, 119.1, 119, 119, 118.9, 116.5, 116.4, 116.4, 116.3, 112, 111.8, 71.1, 67.3, 53.1, 48.4, 45.2, 35.7, 29.2, 29.1, 27.9, 24.2, 21.7.

### Example 10: (R)-1-(3-(dimethylamino)propyl)-6-(2,3,6-trifluorophenyl)-2,5,6,7-tetrahydro-3H-pyrrolo[1,2-c]imidazole-3-thione hydrochloride

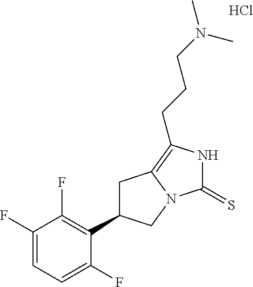 [SMILES: CN(C)CCCc1[nH]c(=S)n2c1C[C@H](c1c(F)ccc(F)c1F)C2 ; Cl]

Compound was prepared from Intermediate 1 and dimethylamine hydrochloride by Method B and isolated as a yellow solid.

¹H NMR (DMSO_{d6}): 11.84 (1H, br s), 10.24 (1H, br s), 7.48 (1H, m), 7.21 (1H, m), 4.45 (1H, quin, J=8.7 Hz), 4.15 (1H, dd, J=11.2, 9.3 Hz), 3.74 (1H, br dd, J=11.4, 8.2 Hz), 3.31 (1H, br dd, J=15.6, 9.2 Hz), 3.00 (2H, m), 2.93 (1H, br dd, J=15.6, 8.4 Hz), 2.72 (6H, m), 2.43 (2H, t, J=7.5 Hz), 1.91 (2H, m).

¹³C NMR (DMSO_{d6}): 156.9, 156.9, 155.3, 155.3, 149.1, 149.1, 149, 149, 147.5, 147.5, 147.4, 147.4, 147.3, 146, 145.9, 145.9, 145.9, 128.1, 118.9, 118.7, 118.1, 116.6, 116.5, 116.4, 116.4, 112, 111.8, 55.7, 48.4, 42, 42, 35.7, 29, 22.5, 21.2.

### Example 11: (R)-1-(3-((R)-3-fluoropyrrolidin-1-yl)propyl)-6-(2,3,6-trifluorophenyl)-2,5,6,7-tetrahydro-3H-pyrrolo[1,2-c]imidazole-3-thione

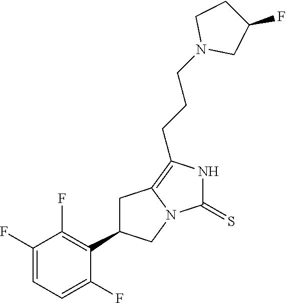 [SMILES: Fc1ccc(F)c([C@H]2Cc3c(CCCN4CC[C@@H](F)C4)[nH]c(=S)n3C2)c1F]

Compound was prepared from Intermediate 1 and (R)-3-fluoropyrrolidine hydrochloride by Method B and isolated as a yellow powder.

¹H NMR (DMSO_{d6}): 11.76 (1H, s), 7.47 (1H, m), 7.17 (1H, m), 5.17 (1H, m), 4.44 (1H, t, J=8.6 Hz), 4.14 (1H, dd, J=11.6, 9.1 Hz), 3.73 (1H, dd, J=11.6, 7.9 Hz), 3.27 (1H, br dd, J=15.6, 9.2 Hz), 2.88 (1H, dd, J=15.6, 8.1 Hz), 2.75 (2H, m), 2.54 (1H, m), 2.37 (4H, m), 2.20 (1H, m), 2.09 (1H, m), 1.83 (1H, m), 1.67 (2H, quin, J=7.3 Hz).

¹³C NMR (DMSO_{d6}): 156.9, 156.9, 156.9, 156.9, 155.3, 155.3, 155.3, 155.3, 155, 149.1, 149, 149, 148.9, 147.5, 147.5, 147.4, 147.4, 147.3, 147.3, 146, 145.9, 145.9, 145.8, 127.7, 119.5, 119.1, 119, 119, 118.9, 116.5, 116.4, 116.4, 116.3, 112, 111.8, 93.9, 92.8, 60.3, 60.1, 54.2, 51.7, 48.4, 35.7, 32.3, 32.2, 29.0, 26.6. 21.8.

### Example 12: (R)-1-(3-(methyl(tetrahydro-2H-pyran-4-yl)amino)propyl)-6-(2,3,6-trifluorophenyl)-2,5,6,7-tetrahydro-3H-pyrrolo[1,2-c]imidazole-3-thione

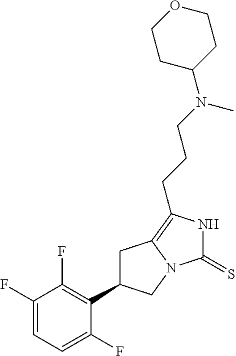 [SMILES: CN(CCCc1[nH]c(=S)n2c1C[C@H](c1c(F)ccc(F)c1F)C2)C1CCOCC1]

Compound was prepared from Intermediate 1 and N-methyltetrahydro-2H-pyran-4-amine by Method C and isolated as a light beige solid.

¹H NMR (DMSO_{d6}): 11.79 (1H, s), 7.47 (1H, m), 7.18 (1H, m), 4.44 (1H, quin, J=8.5 Hz), 4.14 (1H, dd, J=11.4, 9.1 Hz), 3.86 (2H, br d, J=9.5 Hz), 3.73 (1H, dd, J=11.6, 7.9 Hz), 3.28 (1H, dd, J=15.3, 9.2 Hz), 3.24 (2H, m), 2.88 (1H, br dd, J=15.6, 8.1 Hz), 2.50 (1H, br), 2.35 (2H, br), 2.35 (2H, t, J=7.2 Hz), 2.16 (3H, br s), 1.67 (2H, br s), 1.59 (2H, br s), 1.41 (2H, br s).

¹³C NMR (DMSO_{d6}): 156.9, 156.9, 156.9, 155.3, 155.3, 155.3, 155.1, 149.1, 149, 149, 148.9, 147.6, 147.5, 147.5, 147.4, 147.4, 147.3, 147.3, 145.9, 145.9, 145.9, 145.8, 127.6, 119.5, 119.1, 119, 119, 118.9, 116.5, 116.4, 116.4, 116.3, 112, 112, 112, 111.9, 111.8, 111.8, 111.8, 111.8, 66.5, 59.4, 51.9, 48.4, 36.8, 35.6, 29.1, 28.6, 25.4, 21.7.

### Example 13: (6R)-1-(3-(methyl(tetrahydrofuran-3-yl)amino)propyl)-6-(2,3,6-trifluorophenyl)-2,5,6,7-tetrahydro-3H-pyrrolo[1,2-c]imidazole-3-thione hydrochloride

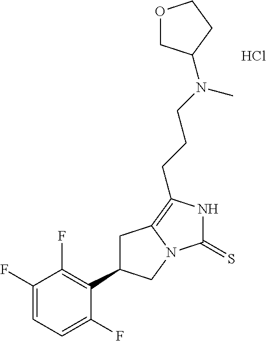 [SMILES: CN(CCCc1[nH]c(=S)n2c1C[C@H](c1c(F)ccc(F)c1F)C2)C1CCOC1 ; Cl]

Compound was prepared from Intermediate 1 and N-methyltetrahydrofuran-3-amine hydrochloride by Method C and isolated as a light yellow solid.

¹H NMR (DMSO_{d6}): 11.84 (1H, m), 10.92 (1H, m), 7.48 (1H, m), 7.20 (1H, m), 4.46 (1H, quin, J=8.6 Hz), 4.15 (1H, dd, J=11.4, 9.3 Hz), 4.05 (1H, m), 4.0 (1H, m), 3.93 (1H, m), 3.74 (2H, m), 3.60 (1H, m), 3.31 (1H, dd, J=15.8, 9.6 Hz), 3.13-2.86 (3H, m), 2.67 (3H, m), 2.43 (2H, m), 2.29-2.10 (2H, m), 1.97 (2H, m).

¹³C NMR (DMSO_{d6}): 157, 156.9, 156.9, 156.9, 155.4, 155.3, 149.1, 149.1, 149, 149, 149, 147.6, 147.5, 147.5, 147.4, 147.4, 147.3, 146, 145.9, 145.9, 145.9, 128, 118.9, 118.8, 118.8, 118.8, 118.7, 118.2, 116.6, 116.5, 116.4, 116.4, 112, 112, 112, 112, 111.9, 111.9, 111.8, 111.8, 67.8, 67.1, 66.9, 66.9, 64.4, 64.1, 59.8, 53.1, 53.1, 52.9, 52.9, 48.4, 37, 37, 36.6, 36.6, 35.7, 29.1, 27.2, 27.2, 26.1, 26, 22.2, 22.2, 22.1, 21.3, 21.3.

### Example 14: (R)-1-(3-(methyl(tetrahydro-2H-pyran-4-yl)amino)propyl)-6-(2,3,6-trifluorophenyl)-2,5,6,7-tetrahydro-3H-pyrrolo[1,2-c]imidazole-3-thione hydrochloride

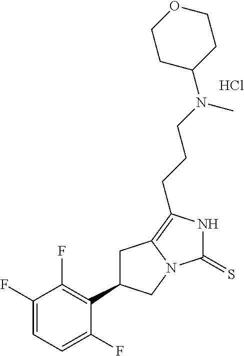 [SMILES: CN(CCCc1[nH]c(=S)n2c1C[C@H](c1c(F)ccc(F)c1F)C2)C1CCOCC1 ; Cl]

Compound was prepared from Intermediate 1 and N-methyltetrahydro-2H-pyran-4-amine by Method C and isolated as a light yellow solid.

¹H NMR (DMSO_{d6}): 11.85 (1H, br s), 10.60 (1H, br s), 7.48 (1H, m), 7.19 (1H, m), 4.46 (1H, quin, J=8.6 Hz), 4.16 (1H, dd, J=11.5, 9.2 Hz), 3.95 (2H, m), 3.74 (1H, br dd, J=11.4, 8.1 Hz), 3.41 (1H, m), 3.31 (3H, m), 3.10 (1H, m), 2.94 (2H, m), 2.67 (3H, d, J=5 Hz), 2.44 (2H, m), 1.98 (3H, m), 1.90 (1H, m), 1.69 (2H, m).

¹³C NMR (DMSO_{d6}): 156.9, 156.9, 156.9, 155.3, 155.3, 155.3, 155.3, 149.1, 149, 149, 149, 148.9, 148.9, 147.6, 147.5, 147.5, 147.4, 147.4, 147.4, 147.3, 147.3, 146, 145.9, 145.9, 145.8, 128.1, 119, 118.9, 118.9, 118.9, 118.9, 118.8, 118.8, 118.7, 118.2, 118.2, 118.2, 116.5, 116.5, 116.4, 116.4, 112, 112, 112, 112, 111.9, 111.8, 111.8, 111.8, 65.5, 65.4, 60.2, 51.1, 48.4, 35.8, 35.7, 35.6, 29.1, 27.1, 26.1, 26.1, 22.1, 22.1, 21.3.

### Example 15: (5aS,6aR)-5a-(3-chloro-2,6-difluorophenyl)-1-(3-morpholinopropyl)-5,5a,6,6a-tetrahydrocyclopropa[3,4]pyrrolo[1,2-c]imidazole-3(2H)-thione hydrochloride

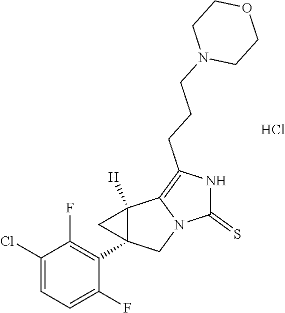 [SMILES: Fc1ccc(Cl)c(F)c1[C@]12C[C@H]1c1c(CCCN3CCOCC3)[nH]c(=S)n1C2 ; Cl]

Compound was prepared from Intermediate 3 and morpholine by Method A and isolated as an off-white solid.

¹H NMR (DMSO_{d6}): 11.80 (1H, s), 10.82 (1H, br s), 7.65 (1H, td, J=8.7, 5.6 Hz), 7.22 (1H, t, J=9.0 Hz), 4.02 (1H, d, J=12.4 Hz), 3.95 (2H, m), 3.78 (2H, br t, J=11.4 Hz), 3.74 (1H, d, J=12.2 Hz), 3.41 (2H, m), 3.07 (4H, m), 2.83 (1H, dd, J=8.3, 4.5 Hz), 2.55 (2H, m), 2.01 (2H, m), 1.67 (1H, dd, J=8.3, 5.5 Hz), 1.32 (1H, t, J=5.0 Hz).

¹³C NMR (DMSO_{d6}): 161.2, 161.2, 159.6, 159.6, 157.8, 157.8, 156.2, 156.1, 156.1, 130.3, 130.3, 118.1, 117.2, 117.1, 116.9, 115.8, 115.8, 115.7, 115.6, 113, 112.8, 63.2, 55.2, 51.3, 51, 51, 26.4, 21.8, 21.7, 21.3, 21.

### Example 16: (5aS,6aR)-5a-(5-chloro-2-fluorophenyl)-1-(3-morpholinopropyl)-5,5a,6,6a-tetrahydrocyclopropa[3,4]pyrrolo[1,2-c]imidazole-3(2H)-thione

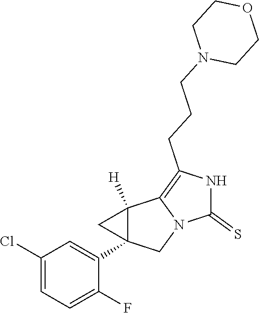 [SMILES: Fc1ccc(Cl)cc1[C@]12C[C@H]1c1c(CCCN3CCOCC3)[nH]c(=S)n1C2]

Compound was prepared from Intermediate 2 and morpholine by Method C and isolated as a white solid.

¹H NMR (DMSO_{d6}): 11.71 (1H, s), 7.48 (1H, dd, J=6.5, 2.7 Hz), 7.42 (1H, ddd, J=8.8, 4.3, 2.7 Hz), 7.29 (1H, dd, J=10.0, 8.9 Hz), 4.06 (1H, d, J=11.8 Hz), 3.77 (1H, d, J=12.2 Hz), 3.57 (4H, br t, J=4.5 Hz), 2.89 (1H, dd, J=8.3, 4.3 Hz), 2.43 (2H, m), 2.34 (4H, m), 2.27 (2H, t, J=7.0 Hz), 1.73 (2H, m), 1.64 (1H, dd, J=8.3, 5.4 Hz), 1.13 (1H, t, J=4.8 Hz).

¹³C NMR (DMSO_{d6}): 161.3, 159.7, 155.7, 130.2, 130.1, 130, 129.3, 129.2, 129, 128.9, 128.3, 128.2, 119.2, 117.5, 117.4, 66.2, 57.1, 53.3, 51.4, 32.3, 24.5, 22.3, 21.8, 20.4.

### Example 17: (5aS,6aR)-5a-(5-chloro-2-fluorophenyl)-1-(3-((tetrahydro-2H-pyran-4-yl)amino)propyl)-5,5a,6,6a-tetrahydrocyclopropa[3,4]pyrrolo[1,2-c]imidazole-3(2H)-thione

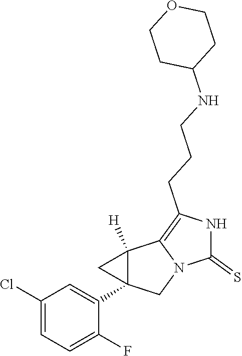 [SMILES: Fc1ccc(Cl)cc1[C@]12C[C@H]1c1c(CCCNC3CCOCC3)[nH]c(=S)n1C2]

Compound was prepared from Intermediate 2 and tetrahydro-2H-pyran-4-amine by Method A and isolated as an off-white solid.

¹H NMR (DMSO_{d6}): 11.74 (1H, m), 7.48 (1H, dd, J=6.5, 2.7 Hz), 7.43 (1H, ddd, J=8.7, 4.4, 2.7 Hz), 7.30 (1H, dd, J=10.0, 8.7 Hz), 4.06 (1H, d, J=12.0 Hz), 3.83 (2H, m), 3.78 (1H, d, J=12.2 Hz), 3.27 (2H, tt, J=11.6, 2.4 Hz), 2.87 (1H, dd, J=8.3, 4.3 Hz), 2.69 (1H, br s), 2.61 (2H, m), 2.47 (2H, td, J=7.4, 2.9 Hz), 1.77 (2H, d, J=13.4 HZ), 1.72 (2H, m), 1.65 (1H, dd, J=8.4, 5.3 Hz), 1.27 (2H, m), 1.13 (1H, t, J=4.8 Hz).

¹³C NMR (DMSO_{d6}): 161.3, 159.7, 155.7, 130.2, 130.1, 130.1, 129.3, 129.3, 129, 128.9, 128.3, 119.1, 117.6, 117.4, 65.8, 53.4, 51.4, 44.5, 32.5, 32.3, 27.7, 22.3, 21.8, 20.5.

### Example 18: (5aS,6aR)-5a-(3-chloro-2,6-difluorophenyl)-1-(3-((tetrahydro-2H-pyran-4-yl)amino)propyl)-5,5a,6,6a-tetrahydrocyclopropa[3,4]pyrrolo[1,2-c]imidazole-3(2H)-thione

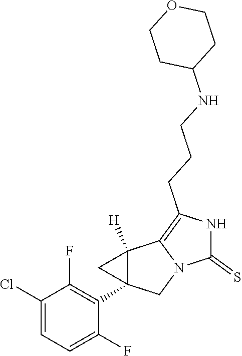 [SMILES: Fc1ccc(Cl)c(F)c1[C@]12C[C@H]1c1c(CCCNC3CCOCC3)[nH]c(=S)n1C2]

Compound was prepared from Intermediate 3 and tetrahydro-2H-pyran-4-amine by Method C and isolated as an off-white solid.

¹H NMR (DMSO_{d6}): 11.77 (1H, m), 7.64 (1H, td, J=8.7, 5.6 Hz), 7.21 (1H, m), 4.01 (1H, d, J=12.2 Hz), 3.83 (2H, m), 3.72 (1H, d, J=12.2 Hz), 3.27 (2H, m), 2.73 (2H, m), 2.63 (2H, m), 2.48 (2H, m), 1.76 (2H, br d, J=12.2 Hz), 1.73 (2H, m), 1.67 (1H, br dd, J=8.3, 5.5 Hz), 1.35-1.20 (3H, m).

¹³C NMR (DMSO_{d6}): 161.3, 161.2, 159.6, 159.6, 157.8, 157.8, 156.2, 156.1, 155.8, 130.3, 130.2, 130, 119.3, 117.3, 117.1, 117, 115.7, 115.6, 113, 112.8, 65.8, 53.4, 51.3, 44.3, 32.2, 27.5, 26.4, 21.8, 21.7, 21.1.

### Example 19: (5aS,6aR)-5a-(3-chloro-2,6-difluorophenyl)-1-(3-(pyrrolidin-1-yl)propyl)-5,5a,6,6a-tetrahydrocyclopropa[3,4]pyrrolo[1,2-c]imidazole-3(2H)-thione

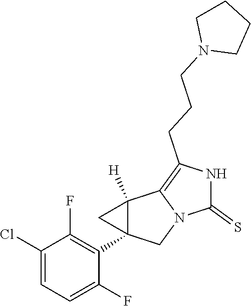 [SMILES: Fc1ccc(Cl)c(F)c1[C@]12C[C@H]1c1c(CCCN3CCCC3)[nH]c(=S)n1C2]

Compound was prepared from Intermediate 3 and pyrrolidine by Method C and isolated as an off-white solid.

¹H NMR (DMSO_{d6}): 11.78 (1H, br s), 7.64 (1H, td, J=8.7, 5.8 Hz), 7.21 (1H, t, J=8.6 Hz), 4.01 (1H, d, J=12.2 Hz), 3.73 (1H, d, J=12.3 Hz), 3.04-2.58 (6H, m br), 2.75 (1H, m), 2.50 (2H, m), 1.93-1.71 (6H, m), 1.67 (1H, br dd, J=8.1, 5.4 Hz), 1.28 (1H, br t, J=5.0 Hz).

¹³C NMR (DMSO_{d6}): 161.3, 161.2, 159.6, 159.6, 157.8, 157.8, 156.2, 156.1, 155.9, 130.3, 130.2, 130.1, 118.9, 117.2, 117.1, 117, 115.7, 115.7, 115.6, 115.6, 112.9, 112.8, 53.9, 53.3, 51.3, 26.4, 25.8, 22.9, 21.7, 21.6, 21.

### Example 20: (5aS,6aR)-5a-(5-chloro-2-fluorophenyl)-1-(3-(pyrrolidin-1-yl)propyl)-5,5a,6,6a-tetrahydrocyclopropa[3,4]pyrrolo[1,2-c]imidazole-3(2H)-thione

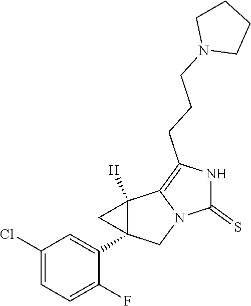 [SMILES: Fc1ccc(Cl)cc1[C@]12C[C@H]1c1c(CCCN3CCCC3)[nH]c(=S)n1C2]

Compound was prepared from Intermediate 2 and pyrrolidine by Method C and isolated as a light beige solid.

¹H NMR (DMSO_{d6}): 11.74 (1H, br s), 7.48 (1H, dd, J=6.5, 2.7 Hz), 7.43 (1H, ddd, J=8.7, 4.3, 2.8 Hz), 7.30 (1H, t, J=9.4 Hz), 4.06 (1H, br d, J=12.0 Hz), 3.78 (1H, d, J=12.0 Hz), 2.90 (1H, br dd, J=8.1, 4.3 Hz), 2.98-2.19 (8H, m), 1.95-1.55 (6H, m), 1.64 (1H, br dd, J=8.2, 5.3 Hz), 1.14 (1H, br t, J=4.7 Hz).

¹³C NMR (DMSO_{d6}): 161.3, 159.7, 155.8, 130.3, 130.1, 130, 129.3, 129.2, 129, 128.9, 128.3, 118.9, 117.6, 117.4, 54.2, 53.4, 51.4, 32.3, 26.2, 22.9, 22.3, 21.8, 20.4.

### Example 21: (5aS,6aR)-5a-(5-chloro-2-fluorophenyl)-1-(3-(((S)-tetrahydro-2H-pyran-3-yl)amino)propyl)-5,5a,6,6a-tetrahydrocyclopropa[3,4]pyrrolo[1,2-c]imidazole-3(2H)-thione

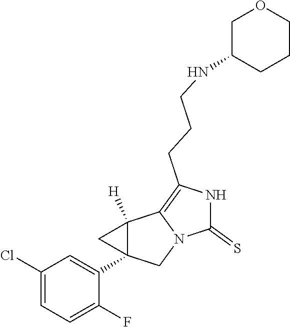 [SMILES: Fc1ccc(Cl)cc1[C@]12C[C@H]1c1c(CCCN[C@H]3CCCOC3)[nH]c(=S)n1C2]

Compound was prepared from Intermediate 2 and (S)-tetrahydro-2H-pyran-3-amine by Method C and isolated as a light beige solid.

¹H NMR (DMSO_{d6}): 11.71 (1H, m), 7.48 (1H, dd, J=6.5, 2.7 Hz), 7.43 (1H, ddd, J=8.7, 4.4, 2.7 Hz), 7.30 (1H, dd, J=10.0, 8.9 Hz), 4.05 (1H, m), 3.80 (1H, m), 3.77 (1H, d, J=12.0 Hz), 3.67 (1H, dt, J=3.5, 11 Hz), 3.24 (1H, br s), 3.01 (1H, m), 2.88 (1H, m), 2.56 (2H, m), 2.50 (1H, m), 2.44 (2H, td, J=7.4, 3.5 Hz), 1.90 (1H, m), 1.69 (2H, br s), 1.64 (1H, dd, J=8.4, 5.3 Hz), 1.59 (1H, m), 1.47 (1H, m), 1.23 (1H, dt, J=7.2, 3.5 Hz), 1.13 (1H, m).

¹³C NMR (DMSO_{d6}): 161.3, 159.7, 155.7, 130.1, 130, 130, 129.3, 129.2, 129, 128.9, 128.3, 119.3, 117.5, 117.4, 71.6, 67.3, 53.3, 51.3, 45.4, 32.3, 29.7, 28.4, 24.4, 22.3, 21.8, 20.5.

### Example 22: (5aS,6aR)-5a-(3-chloro-2,6-difluorophenyl)-1-(3-(((S)-tetrahydro-2H-pyran-3-yl)amino)propyl)-5,5a,6,6a-tetrahydrocyclopropa[3,4]pyrrolo[1,2-c]imidazole-3(2H)-thione

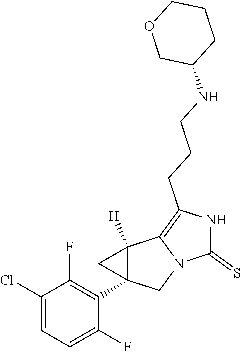 [SMILES: Fc1ccc(Cl)c(F)c1[C@]12C[C@H]1c1c(CCCN[C@H]3CCCOC3)[nH]c(=S)n1C2]

Compound was prepared from Intermediate 3 and (S)-tetrahydro-2H-pyran-3-amine by Method C and isolated as a light yellow solid.

¹H NMR (DMSO_{d6}): 11.74 (1H, m), 7.63 (1H, td, J=8.7, 5.7 Hz), 7.20 (1H, t, J=9.0 Hz), 4.01 (1H, d, J=12.2 Hz), 3.78 (1H, dt, J=10.7, 1.9 Hz), 3.72 (1H, d, J=12.2 Hz), 3.67 (1H, dt, J=7.4, 3.4 Hz), 3.22 (1H, td, J=10.9, 2.5 Hz), 2.97 (1H, br t, J=9.8 Hz), 2.72 (1H, dd, J=8.2, 4.4 Hz), 2.54 (2H, m), 2.48 (1H, m), 2.45 (2H, m), 1.90 (1H, m), 1.66 (3H, m), 1.58 (1H, m), 1.44 (1H, m), 1.31-1.15 (2H, m).

¹³C NMR (DMSO_{d6}): 161.3, 161.2, 159.6, 159.6, 157.8, 157.8, 156.2, 156.1, 155.7, 130.3, 130.2, 129.9, 119.6, 117.3, 117.1, 117, 115.7, 115.7, 115.6, 115.6, 112.9, 112.8, 71.7, 67.3, 53.4, 51.2, 45.3, 29.7, 28.5, 26.3, 24.4, 21.8, 21.7, 21.1

### G. Dopamine-β-Hydroxylase Inhibition Assay

The ability of a compound to inhibit DβH activity may be assessed in human plasma using the following assay. Preferred compounds of the present invention (including most of the specific Examples above) exhibit activity in “% of control” of ≤50% at 0.1 μM in this cell assay. More preferred compounds of the present invention exhibit activity in “% of control” of ≤20% at 0.1 μM in this cell assay. Especially preferred compounds of the present invention exhibit an IC₅₀ of ≤20 nM in this assay.

Dopamine beta hydroxylase activity in human plasma was measured by the method of Nagatsu and Udenfriend (Nagatsu, T. and Udenfriend, S. “Photometric assay of dopamine-p-hydroxylase activity in human blood.” Clin. Chem. 18(9) 980-983, 1972) with minor modifications. Catalase, N-ethylmaleimide, tyramine, disodium fumarate, pargyline, sodium acetate, ascorbic acid, copper sulfate and octopamine were obtained from Sigma Chemical Co., St. Louis, Mo. 63178. Human plasma samples were obtained from healthy donors (Instituto Português do Sangue Transplantação, Centro Sangue Transplantação, Porto, Portugal). From date of collection, plasma was stored at −80° C. until use. Test compounds were initially prepared in dimethyl sulfoxide at a concentration of 10 mM and diluted in dimethyl sulfoxide to the required concentrations. Test compounds were further diluted in ultrapure water to a concentration 20-fold to that of the final concentration to be tested. Final concentrations of test compounds were 10 and 100 nM. The various reagents used to make up the incubation buffer were premixed and consisted of the following components: sodium acetate buffer (1 M, pH 5.0, 18 mL), sodium fumarate (0.2 M, 4.5 mL), ascorbic acid (0.2 M, 4.5 ml, freshly prepared), pargyline (20 mM, freshly prepared, 4.5 mL), N-ethylmaleimide (0.2 M, 4.5 mL), catalase (10 000 U/mL, 9 mL), copper sulfate (20 μM, 4.5 mL) and 4.5 ultrapure water. The standard incubation mixture (total volume, 950 μL) contained: 50 μL of compound or vehicle (dimethyl sulfoxide 2%); 700 μL of incubation buffer; 125 μL of plasma (or saline for blank reaction or standard curve); 75 μL of saline. The reaction mixture was placed in water bath, shaking at 37° C. and pre-incubated for 10 minutes. Tyramine (0.5 M) was added and incubation proceeded for 45 minutes. The reaction contents were exposed to air. A sample of enzyme preparation (with 125 μL of plasma) that had been added perchloric acid 2 M at the end of the pre-incubation period was used as blank. A blank for each of the tested compounds was used. For octopamine standard curve, perchloric acid 2 M was replaced by increasing concentrations of octopamine prepared in perchloric acid 2 M (0.5, 1, 2.5, 5, 7.5, 10, 15, 20 μg/mL, final concentration). The incubation was stopped by adding 200 μL of 2 M molar perchloric acid, and the mixture was centrifuged at 9000 g for 5 min. The supernatant fluid (800 μL) was transferred to a column (SPE cartridge ISOLUTE SCX-3, 100 mg) and centrifuged at 150 g for 2 min. The column was washed two more times with 0.5 ml of ultrapure water by centrifuging at 150 g for 2 min. The adsorbed octopamine was eluted twice with 0.3 mL of 4 M ammonium hydroxide by centrifuging at 150 g for 2 min. Octopamine in the eluate was then converted to p-hydroxybenzaldehyde by adding 200 μL of sodium periodate (2%) and incubating for 6 min. Excess periodate was than reduced by adding 200 μL of sodium metabisulfite (10%). Absorbance was measured at 330 mm in a 96-well plate by use of a SpectraMAX plus 384 (Molecular Devices) with software SOFTmax® PRO Software 5.3 spectrophotometer. Absorbance was linear with octopamine concentration from 0.5 to 20 μg/mL. Dopamine beta hydroxylase activity is determined as nmol of octopamine formed/mL of plasma/hour and effect of compounds is presented as % control. Results are reported in the table below as activity in % of control at the inhibitor concentration tested.

IC₅₀ values of selected compounds were calculated based on curve fitting of results of 6 different compound concentrations (100 nM to 0.3 nM). IC₅₀ data are reported in nM concentration.

### H. Catecholamine Determination

Catecholamines quantification in brain stem and heart left ventricle was performed as previously described (Bonifacio, M. J.; Sousa, F.; Neves, M.; Palma, N.; Igreja, B.; Pires, N. M.; Wright, L. C.; Soares-da-Silva, P. “Characterization of the interaction of the novel anthyhypertensive etamicastat with human dopamine-beta-hydroxylase: comparison with nepicastat.” Eur. J. Pharmacol. 751, 50-58, 2015) with minor modifications. Test compounds were prepared in 40% of kleptose at a concentration of 2.5 mg/ml to be administered at a dose of 10 mg/kg. Compounds and vehicle (kleptose 400%) were administered to Wistar rats and tissues (brain stem or heart left ventricle) collected in perchloric acid (0.2M) at defined time points after administration. Tissues were stored overnight at 4° C. and the solution was then filtered by centrifugation (1500 g, 4 min, 4° C.) through 0.22 μm pore size filters (Costar Spin-x from Corning Inc., USA). Catecholamines were quantified in filtrates by directly injecting 50 μl of sample volume on a HPLC system with electrochemical detection, using a Spheri-5RP-185 mm column (Perkin-Elmer). Mobile phase consisted of a solution containing 0.1M citric acid, 0.1M sodium acetate, 0.15 mM EDTA, 1 mM dibutylamine, 1 mM octylsulfate, and 53 methanol adjusted to pH 3.5 with perchloric acid.

### L. Biological Data

TABLE 1
The following table shows DβH inhibition and IC₅₀
values in human plasma for the compounds:

| Example | DβH (0.1 μM)^{a} | DβH (0.01 μM)^{a} | IC₅₀ (nM)^{b} |
|---|---|---|---|
| 1 | 12.9 | 64.6 | 12.7 |
| 2 | 66.6 | 98.4 | ND |
| 3 | 15.5 | 57.3 | 9.5 |
| 4 | 54.2 | 82.7 | ND |
| 5 | 5.4 | 42.5 | 12.7 |
| 6 | 10.4 | 63.6 | 11.6 |
| 7 | 3.0 | 42.5 | 8.3 |
| 8 | 52.8 | 113.4 | ND |
| 9 | 12.2 | 48.1 | 19.1 |
| 10 | 45.8 | 86.2 | ND |
| 11 | 7.7 | 40.6 | 7.2 |
| 12 | 18.9 | 71.2 | 20.9 |
| 13 | 29.3 | 69.9 | 22.2 |
| 14 | 19.0 | 73.2 | 33.1 |
| 15 | 1.0 | 13.6 | 2.7 |
| 16 | 3.3 | 36.2 | 7.7 |
| 17 | 3.9 | 41.8 | 9.9 |
| 18 | 1.0 | 20.1 | 4.0 |
| 19 | 1.0 | 17.0 | 3.0 |
| 20 | 9.1 | 53.4 | 9.8 |
| 21 | 8.5 | 45.3 | ND |
| 22 | 3.5 | 23.5 | 4.0 |

^{a}% of Control;
^{b}IC₅₀ values are presented with 95% of confidence intervals

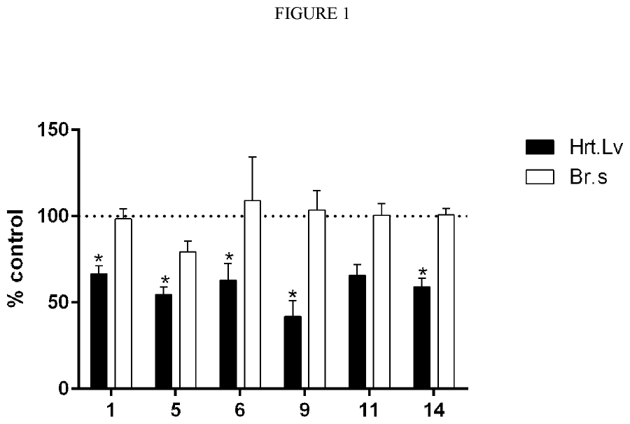

FIG. 1 shows levels of noradrenaline (NA) in brain stem (Br.s) and heart left ventricle (Hrt.lv) at 15 h post-dose after oral administration of 10 mg/kg of compounds 1, 5, 6, 9, 11 and 14. Data are presented as % of Control. Each column represents mean±SEM of 4 to 5 rats per group.

As can be seen from FIG. 1, the compounds are peripherally selective, i.e. they reduce the levels of NA in Hrt.lv (significantly different from corresponding control values (*P<0.05)), meanwhile the levels of NA in Br.s remain unaltered (not significantly different from corresponding control values (*P>0.05)). The Kruskal-Wallis test followed by Dunn's multiple comparisons test was used for statistical analysis.

1. A compound of formula I, or a pharmaceutically acceptable salt or solvate thereof: 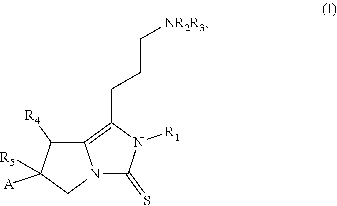
wherein:
R₁ is hydrogen;
R₂ is hydrogen; and
R₃ is hydrogen, methyl, 6-membered heterocyclyl, or CH₂X wherein X is 5- or 6-membered heterocyclyl; or
R₂ is methyl; and
R₃ is methyl, 5- or 6-membered heterocyclyl, or CH₂X wherein X is 5- or 6-membered heterocyclyl; or
R₂ and R₃ combine, together with the N atom to which they are attached, to form a 5- or 6-membered N-heterocyclyl optionally substituted with one fluoro substituent; R₄ is hydrogen; and
R₅ is hydrogen; or
R₄ and R₅ combine, together with the carbon atoms to which they are attached, to form a cyclopropyl ring; and
A is 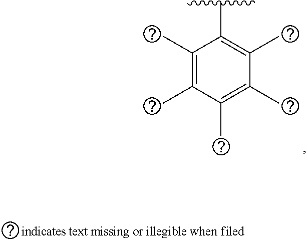
wherein:
X₁ is hydrogen or halo;
X₁′ is hydrogen or halo;
X₂ is hydrogen or halo;
X₂′ is hydrogen or halo; and
X₃ is hydrogen;
with the proviso that the compounds
(R)-1-(3-(pyrrolidin-1-yl)propyl)-6-(2,3,5,6-tetrafluorophenyl)-2,5,6,7-tetrahydro-3H-pyrrolo[1,2-c]imidazole-3-thione hydrochloride,
(R)-1-(3-(pyrrolidin-1-yl)propyl)-6-(2,3,5,6-tetrafluorophenyl)-2,5,6,7-tetrahydro-3H-pyrrolo[1,2-c]imidazole-3-thione, and
(R)-1-(3-(pyrrolidin-1-yl)propyl)-6-(2,3,6-trifluorophenyl)-2,5,6,7-tetrahydro-3H-pyrrolo[1,2-c]imidazole-3-thione hydrofluoride
are excluded.

2. (canceled)

3. A compound according to claim 10, wherein:
R₂ is hydrogen;
R₃ is hydrogen, methyl, 6-membered heterocyclyl, or CH₂X wherein X is 6-membered heterocyclyl.

4. A compound according to claim 1, wherein:
R₂ is methyl;
R₅ is methyl, 5- or 6-membered heterocyclyl, or CH₂X wherein X is 6-membered heterocyclyl.

5. A compound according to claim 3, wherein:
R₂ is hydrogen;
R₃ is hydrogen, methyl, tetrahydropyranyl, or CH₂X wherein X is tetrahydropyranyl.

6. A compound according to claim 4, wherein:
R₂ is methyl;
R₅ is methyl, tetrahydrofuranyl, tetrahydropyranyl, or CH₂X wherein X is tetrahydropyranyl

7. A compound according to claim 1, wherein:
R₂ and R₅ combine together with the N atom to which they are attached to form a 5- or 6-membered N-heterocyclyl optionally substituted with one fluoro substituent.

8. A compound according to claim 7, wherein:
R₂ and R₅ combine together with the N atom to which they are attached to form a pyrrolidinyl, 3-fluoropyrrolidinyl, piperidinyl or morpholinyl group.

9. A compound according to claim 1, wherein:
R₁ and R₅ are both hydrogen.

10. A compound according to claim 1, wherein:
R₄ and R₅ combine, together with the carbon atom to which they are attached, to form a cyclopropyl ring.

11. A compound according to claim 1, wherein:
A is, 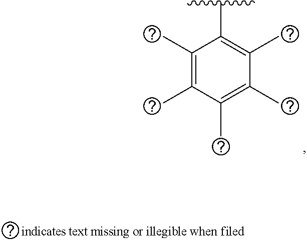
wherein:
X₁ is hydrogen, fluoro or chloro;
X₁′ is hydrogen, fluoro or chloro;
X₂ is hydrogen, fluoro, chloro or bromo;
X₂′ is hydrogen, fluoro, chloro or bromo;
X₃ is hydrogen.

12. A compound according to claim 11, wherein:
X₁ is hydrogen or fluoro;
X₁′ is fluoro;
X₂ is fluoro or chloro;
X₂′ is hydrogen;
X₃ is hydrogen.

13. A compound according to claim 1, wherein more than 50% of substituents R₅ and A have the stereochemical configuration of formula Ic:

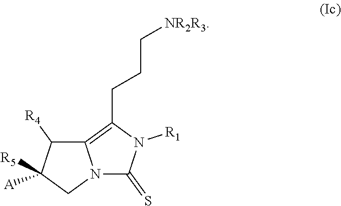

14. A compound according to claim 1, wherein more than 50% of substituents R₅ and A have the stereochemical configuration of formula Id:

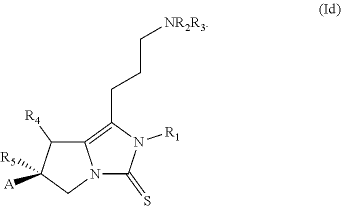

15. A compound according to claim 1, wherein the compound corresponds to formula Ie: 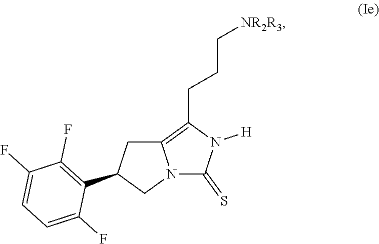
wherein:
R₂ is hydrogen; and
R₄ is hydrogen, methyl, 6-membered heterocyclyl, or CH₂X wherein X is a 5- or 6-membered heterocyclyl; or
R₂ is methyl; and
R₃ is methyl, 5- or 6-membered heterocyclyl, or CH₂X wherein X is a 5- or 6-membered heterocyclyl; or
R₂ and R₃ combine together with the N atom to which they are attached to form a 5- or 6-membered N-heterocyclyl optionally substituted with one fluoro substituent.

16. A compound according to claim 15, or a pharmaceutically acceptable salt or solvate thereof, wherein:
R₂ is hydrogen; and
R₄ is hydrogen, methyl, tetrahydropyranyl, or CH₂X wherein X is tetrahydrofuranyl or tetrahydropyranyl; or
R₂ is methyl; and
R₃ is methyl, tetrahydrofuranyl, tetrahydropyranyl, or CH₂X wherein X is tetrahydrofuranyl or tetrahydropyranyl; or
R₂ and R₃ combine together with the N atom to which they are attached to form a 3-fluoropyrrolidinyl or morpholinyl group.

17. A compound according to claim 1, wherein the compound corresponds to formula If: 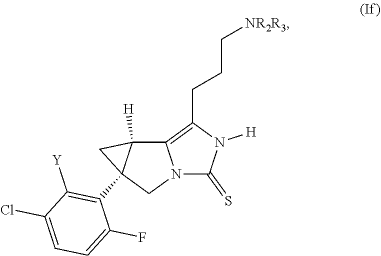
wherein:
R₂ is hydrogen; and
R₃ is 6-membered heterocyclyl; or
R₂ and R₃ combine together with the N atom to which they are attached to form a 5- or 6-membered N-heterocyclyl; and
Y is hydrogen or fluoro.

18. A compound according to claim 17, wherein:
R₂ is hydrogen; and
R₃ is tetrahydropyranyl; or
R₂ and R₃ combine together with the N atom to which they are attached to form a pyrrolidinyl or morpholinyl.

19-21. (canceled)

22. A method for treating or preventing at least one condition ameliorated by inhibition of dopamine-beta-hydroxylase outside the central nervous system, the method comprising administering a therapeutically effective amount of a compound according to claim 1, or a pharmaceutically acceptable salt thereof, to a patient in need thereof.

23. A pharmaceutical composition, comprising (i) a therapeutically effective amount of a compound according to claim 1, or a pharmaceutically acceptable salt thereof, and (ii) a pharmaceutically acceptable excipient.
